(19) United States
(12) Patent Application Publication
(10) Pub. No.: US 2013/0035537 A1
(43) Pub. Date: Feb. 7, 2013

(54) ROBOTIC SYSTEMS AND METHODS FOR TREATING TISSUE

(71) Applicants: Daniel T. Wallace, Santa Cruz, CA (US); Dale Bergman, Cupertino, CA (US); Ruchi Choksi, San Jose, CA (US); Aaron Grogan, Scotts Valley, CA (US)

(72) Inventors: Daniel T. Wallace, Santa Cruz, CA (US); Dale Bergman, Cupertino, CA (US); Ruchi Choksi, San Jose, CA (US); Aaron Grogan, Scotts Valley, CA (US)

(21) Appl. No.: 13/310,596

(22) Filed: Dec. 2, 2011

Related U.S. Application Data
(60) Provisional application No. 61/515,744, filed on Aug. 5, 2011.

(51) Int. Cl. A61M 25/092; A61M 36/12

(52) U.S. Cl. 600/8; 604/95.01; 606/33; 604/500

(57) ABSTRACT

A method of manipulating an elongate member in at least two degrees of freedom includes holding an elongate member between two rotary members that define respective rotational axes, the elongate member having a flexible proximal portion, a distal rigid needle attached to the proximal portion, and an operative element for delivering energy, the needle having a distal port, actuating at least one of the rotary members in a rotational direction about its rotational axis to generate a corresponding linear motion of the elongate member along a longitudinal axis of the elongate member, and actuating at least one of the rotary members in a linear direction along its rotational axis to generate a corresponding rotational motion of the elongate member about the longitudinal axis of the elongate member.

### RELATED APPLICATION DATA

This application claims priority to, and the benefit of, U.S. Provisional Patent Application No. 61/515,744, filed Aug. 5, 2011, pending, the entire disclosure of which is expressly incorporated by reference herein.

### INCORPORATION BY REFERENCE

All of the following U.S. patent applications are expressly incorporated by reference herein for all purposes:
U.S. patent application Ser. No. 13/173,994, filed on Jun. 30, 2011,
U.S. patent application Ser. No. 11/179,007, filed on Jul. 6, 2005,
U.S. patent application Ser. No. 12/079,500, filed on Mar. 26, 2008,
U.S. patent application Ser. No. 11/678,001, filed on Feb. 22, 2007,
U.S. Patent Application No. 60/801,355, filed on May 17, 2006,
U.S. patent application Ser. No. 11/804,585, filed on May 17, 2007,
U.S. patent application Ser. No. 11/640,099, filed on Dec. 14, 2006,
U.S. patent application Ser. No. 12/507,727, filed on Jul. 22, 2009,
U.S. patent application Ser. No. 12/106,254, filed on Apr. 18, 2008,
U.S. patent application Ser. No. 12/192,033, filed on Aug. 14, 2008,
U.S. patent application Ser. No. 12/236,478, filed on Sep. 23, 2008,
U.S. patent application Ser. No. 12/833,935, filed on Jul. 9, 2010,
U.S. patent application Ser. No. 12/822,876, filed on Jun. 24, 2010,
U.S. patent application Ser. No. 12/614,349, filed on Nov. 6, 2009,
U.S. patent application Ser. No. 11/690,116, filed Mar. 22, 2007,
U.S. patent application Ser. No. 11/176,598, filed Jul. 6, 2005,
U.S. patent application Ser. No. 12/012,795, filed Feb. 1, 2008,
U.S. patent application Ser. No. 12/837,440, Jul. 15, 2010,
U.S. Patent Application No. 61/513,488, filed Jul. 8, 2011, and
U.S. patent application Ser. No. 13/174,605, filed June 30.

### FIELD

The application relates generally to robotically controlled surgical systems, and more particularly to flexible instruments and instrument drivers that are responsive to a master controller for performing surgical procedures to treat tissue, such as tissue in the livers.

### BACKGROUND

Liver tumors may be treated by resection through open surgery procedures. In some cases, liver tumors may also be treated using radiofrequecy ablation. Ablation procedures may be performed through open surgery, which permits the surgeon's hands access to internal organs. Ablation procedures may also be performed percutaneously by inserting a rigid ablation probe through a patient's skin to reach the liver underneath the skin. However, such technique may not allow certain liver tissue, such as tissue at the lobus quadratus or the lobus spigelii, to be reached.

### SUMMARY

The subject application describes, among other things, a robotic system for controlling an elongate instrument. By means of non-limiting examples, the elongate instrument may include a needle configured to deliver energy to treat tissue (e.g., liver tissue). The energy may be radiofrequency energy, heat, ultrasound energy, or any of other forms of energy. In some embodiments, the needle may optionally include a distal port and/or side ports for delivering fluid to control energy delivery to the tissue. Alternatively, or additionally, the distal port and/or the side ports may also be used to deliver other substance, such as an agent, a drug, embolic materials, radioactive seeds, etc., to a target site. Also, in some embodiments, the robotic system may optionally include a catheter surrounding at least a portion of the elongate instrument, and a sheath surrounding at least a part of the catheter. In some embodiments, the sheath may be considered a catheter itself. The catheter and/or the sheath may be placed in a vessel, and may be steerable in some embodiments to assist placement of the elongate instrument at a desired target location, such as the liver. Also, in some embodiments, the catheter and/or the sheath may be coupled to a drive assembly of the robotic system, which robotically moves the catheter and/or the sheath.

In accordance with some embodiments, a robotic system includes an elongate member comprising a flexible proximal portion, a distal rigid needle attached to the proximal portion, and an operative element for treating tissue, the needle having a distal port, and an elongate member holder having first and second rotary members configured to hold and manipulate the proximal portion of the elongate member, wherein the first rotary member defines a first rotational axis, and the second rotary member defines a second rotational axis, wherein the first and second rotary members are moveable relative to each other in opposite rotational directions about their respective axes to generate a corresponding linear motion of the elongate member along a longitudinal axis of the elongate member when the elongate member is held by the rotary members, and wherein at least one of the first and second rotary members is moveable in a linear direction along its rotational axis to generate a corresponding rotational motion of the elongate member about the longitudinal axis of the elongate member when the elongate member is held by the rotary members.

In accordance with other embodiments, a method of manipulating an elongate member in at least two degrees of freedom includes holding an elongate member between two rotary members that define respective rotational axes, the elongate member having a flexible proximal portion, a distal rigid needle attached to the proximal portion, and an operative element for delivering energy, the needle having a distal port, actuating at least one of the rotary members in a rotational direction about its rotational axis to generate a corresponding linear motion of the elongate member along a longitudinal axis of the elongate member, and actuating at least one of the rotary members in a linear direction along its rotational axis to generate a corresponding rotational motion of the elongate member about the longitudinal axis of the elongate member.

Other and further aspects and features will be evident from reading the following detailed description of the embodiments.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates a robotic surgical system in which apparatus, system and method embodiments may be implemented.

FIG. 2 illustrates how the adapter base plate assembly is utilized to attach a support assembly and instrument driver to an operating table or surgical bed.

FIG. 3 a sheath and guide catheter assembly, and an elongate member manipulator mounted on an instrument driver

FIG. 4 illustrates an example of an operator workstation of the robotic surgical system shown in FIG. 1 with which a catheter instrument can be manipulated using different user interfaces and controls.

FIG. 5A further illustrates the instrument driver shown in FIG. 3 without the elongate member manipulator mounted on an instrument driver.

FIG. 5B further illustrates the instrument driver shown in FIG. 5A without the sheath and guide catheter assembly.

FIG. 5C further illustrates the instrument driver shown in FIG. 5B with skins removed.

FIGS. 6A and 6B illustrate a sheath and guide catheter assembly positioned over respective sterile adaptors and mounting plates from top and bottom perspectives respectively

FIGS. 7A and 7B illustrate top and bottom perspectives respectively of a portion of an instrument driver with a sheath splayer positioned over a sterile adaptor.

FIG. 7C illustrates an exploded view of the sheath splayer shown in FIG. 7A without a purge tube.

FIG. 7D illustrates top and bottom views of a pulley assembly positioned over a floating shaft.

FIG. 7E illustrates the floating shaft of FIG. 7D installed and un-installed onto a sleeve receptacle.

FIG. 8 illustrates a guide carriage of the instrument driver shown in FIG. 5C with pulleys and guide articulation motors.

FIG. 9 is a perspective view of a slidable carriage or funicular assembly of an instrument driver and sleeve receptacles configured to receive and engage with floating shafts.

FIG. 10 illustrates a sheath block, sheath insert motor, guide insert motor and leadscrews removed from the instrument driver shown in FIG. 5C.

FIGS. 10A and 10B illustrate different perspective views of the sheath block with sheath output plate positioned over receptacle sleeves.

FIG. 10C illustrates sheath articulation motors coupled to motor driven interfaces and receptacle sleeves.

FIGS. 11A-11H illustrate side and cross-sectional views of a catheter bent in various configurations with pull wire manipulation.

FIG. 12 illustrates an open loop control model.

FIG. 13 illustrates a control system in accordance with some embodiments.

FIG. 14 illustrates a user interface for a master input device.

FIG. 15 illustrates an elongate member in accordance with some embodiments.

FIG. 16 illustrates another elongate member in accordance with other embodiments.

FIGS. 17A-17D illustrates different elongate member manipulators in accordance with different embodiments.

FIG. 18A illustrates a front perspective view of a variation of an elongate member manipulator.

FIG. 18B illustrates an end perspective view of the elongate member manipulator of FIG. 18A.

FIG. 18C illustrates a cross sectional view of the elongate member manipulator of FIG. 18A.

FIG. 18D illustrates a top cross sectional view of the elongate member manipulator of FIG. 18A.

FIGS. 19A-19B are schematic illustrations showing top and front views of feed rollers actuating an elongate member.

FIG. 20 illustrates a cross sectional view of one variation of a roller actuator.

FIG. 21 illustrates a cross sectional view of one variation of a feed roller with a drape,

FIG. 22A illustrates a perspective view of the instrument driver, the guide splayer and a variation of an elongate member manipulator.

FIG. 22B illustrates a closer view of the instrument driver, the elongate member manipulator, and the guide splayer of FIG. 22A.

FIG. 23 illustrates a perspective view of the elongate member manipulator of FIG. 22A, showing the manipulator in an open configuration and mounted to a manipulator mounting bracket.

FIG. 24A illustrates the elongate member manipulator of FIG. 23, showing the manipulator in a closed configuration.

FIGS. 24B-24C illustrate the elongate member manipulator of FIG. 23 with an idler belt assembly removed, showing the manipulator open by varying degrees.

FIG. 24D illustrates the elongate member manipulator of FIG. 24B in a closed configuration.

FIG. 24E illustrates a cross-sectional view of the elongate member manipulator of FIG. 24A.

FIG. 25A illustrates a back view of the elongate member manipulator of FIG. 23.

FIGS. 25B-25C illustrates various perspective views of the elongate member manipulator.

FIG. 26A illustrates a side view of the elongate member manipulator, showing a hinge mechanism in a closed configuration.

FIG. 26B illustrates the elongate member manipulator of FIG. 26A, showing the hinge mechanism in an open configuration.

FIG. 26C illustrates a cross sectional perspective view of the elongate member manipulator.

FIG. 27A illustrates driving mode(s) in accordance with some embodiments.

FIG. 27B illustrates driving mode(s) in accordance with other embodiments.

FIG. 27C illustrates driving mode(s) in accordance with other embodiments.

FIG. 27D illustrates driving mode(s) in accordance with other embodiments.

FIG. 28A-28F illustrates a method of using a robotic system to treat tissue in accordance with some embodiments.

### DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

I. Robotic Surgical Systems

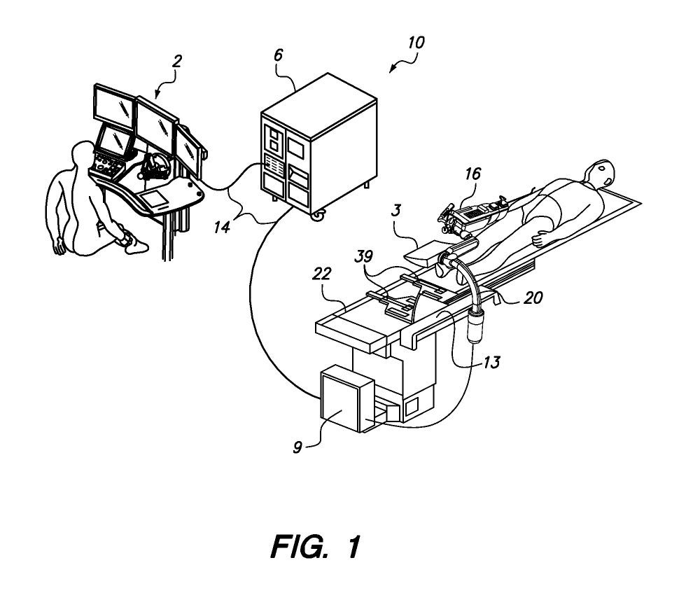

Embodiments described herein generally relate to apparatus, systems and methods for robotic surgical systems. A robotic surgical system in which embodiments described herein may be implemented is described with reference to FIGS. 1-10C.

Referring to FIG. 1, a robotically controlled surgical system 10 in which embodiments of apparatus, system and method may be implemented includes an operator workstation 2, an electronics rack 6 and associated bedside electronics box 9, a setup joint or support assembly 20 (generally referred to as “support assembly”), and a robotic instrument driver 16 (generally referred to as “instrument driver”). A surgeon is seated at the operator workstation 2 and can monitor the surgical procedure, patient vitals, and control one or more robotic surgical devices.

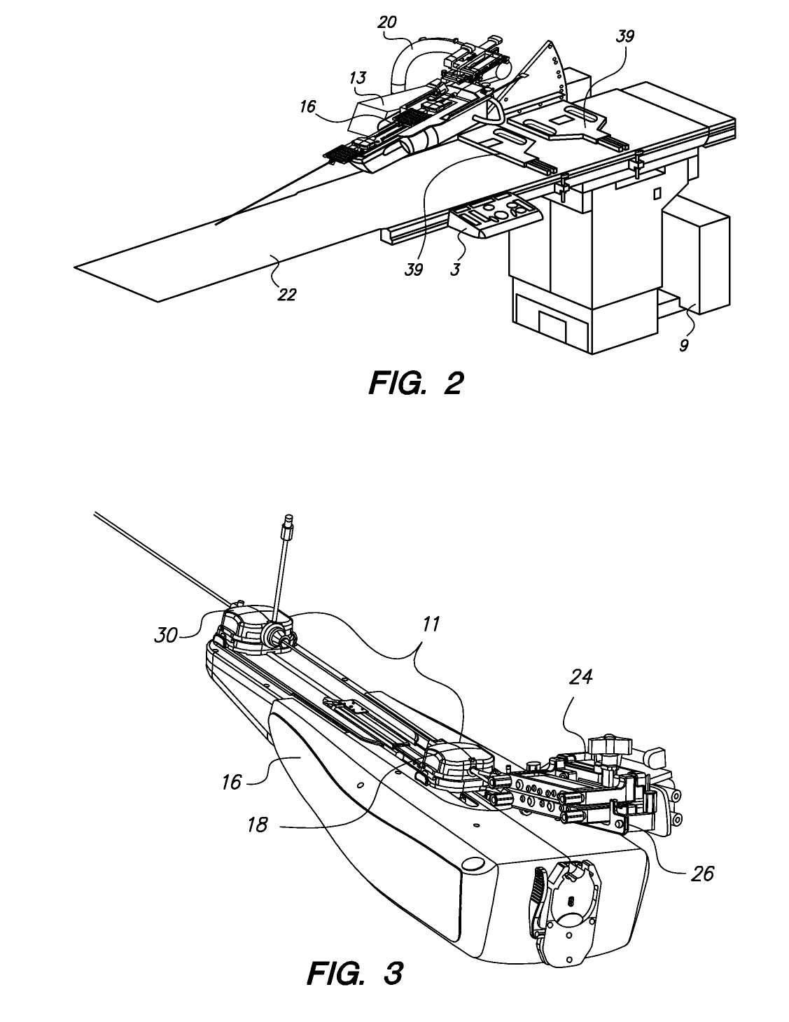

Referring to FIG. 2, the instrument driver 16, setup joint mounting brace 20, and bedside electronics box are shown in greater detail. Referring to FIG. 3, the instrument driver 16 is illustrated including an elongate member manipulator 24 and a robotic catheter assembly 11 installed. The robotic catheter assembly 11 includes a first or outer robotic steerable complement, otherwise referred to as a sheath instrument 30 (generally referred to as “sheath” or “sheath instrument”) and/or a second or inner steerable component, otherwise referred to as a robotic catheter or guide or catheter instrument 18 (generally referred to as “catheter” or “catheter instrument”). The sheath instrument 30 and catheter instrument 18 are controllable using the instrument driver 16. During use, a patient is positioned on an operating table or surgical bed 22 (generally referred to as “operating table”) to which the support assembly 20, instrument driver 16, and robotic catheter assembly 11 are coupled or mounted.

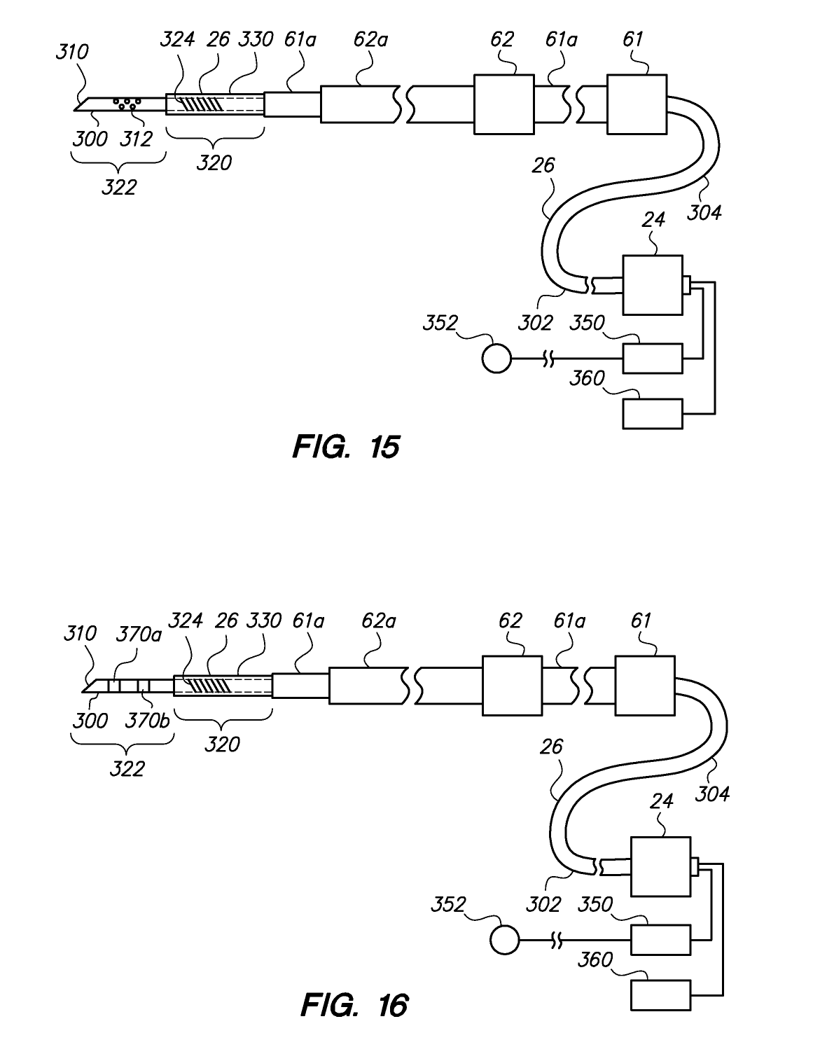

In the illustrated embodiments, the elongate member manipulator 24 (generally referred to as “manipulator”) is configured for manipulating an elongate member 26 (which will be described in further detail with reference to FIG. 15). The elongate member 26 is configured to deliver energy to treat tissue, such as tissue at a liver. During use, at least a part of the elongate member 26 is disposed within a lumen of the catheter instrument 18, and the proximal end of the elongate member 26 is removably coupled to the manipulator 24. In some embodiments, the manipulator 24 is configured to advance and retract the elongate member 26 relative to the catheter instrument 18. In other embodiments, the manipulator 24 may also be configured to roll the elongate member 26 so that it rotates about its longitudinal axis. Embodiments of the elongate member 26 and the manipulator 24 will be described in detail below.

Various system components in which embodiments described herein may be implemented are illustrated in close proximity to each other in FIG. 1, but embodiments may also be implemented in systems 10 in which components are separated from each other, e.g., located in separate rooms. For example, the instrument driver 16, operating table 22, and bedside electronics box 9 may be located in the surgical area with the patient, and the operator workstation 2 and the electronics rack 6 may be located outside of the surgical area and behind a shielded partition. System 10 components may also communicate with other system 10 components via a network to allow for remote surgical procedures during which the surgeon may be located at a different location, e.g., in a different building or at a different hospital utilizing a communication link transfers signals between the operator control station 2 and the instrument driver 16. System 10 components may also be coupled together via a plurality of cables or other suitable connectors 14 to provide for data communication, or one or more components may be equipped with wireless communication components to reduce or eliminate cables 14. In this manner, a surgeon or other operator may control a surgical instrument while being located away from or remotely from radiation sources, thereby decreasing the operator's exposure to radiation.

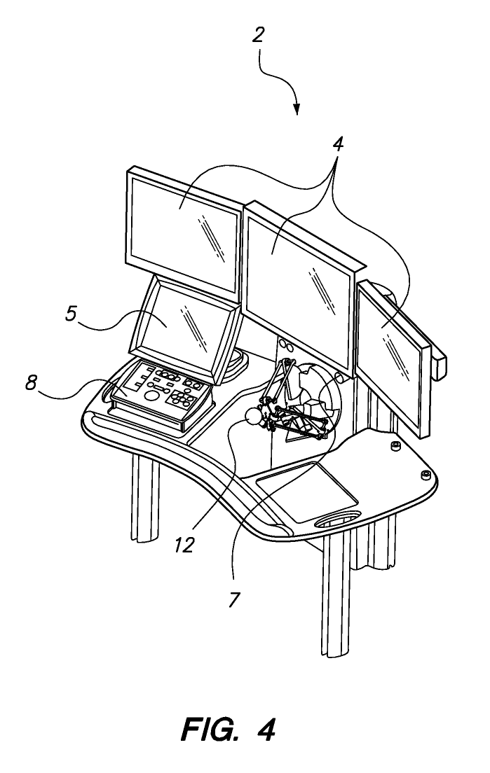

Referring to FIG. 4, one example of an operator workstation 2 that may be used with the system 10 shown in FIG. 1 includes three display screens 4, a touch screen user interface 5, a control button console or pendant 8, and a master input device (MID) 12. The MID 12 and pendant 8 serve as user interfaces through which the surgeon can control operation of the instrument driver 16 and attached instruments. By manipulating the pendant 8 and the MID 12, a surgeon or other operator can cause the instrument driver 16 to remotely control the catheter instrument 18 and/or the sheath instrument 30 mounted thereon. Also, in some embodiments, by manipulating one or more controls at the station 2, the surgeon or operator may cause the manipulator 24 to remotely move the elongate member 26. A switch 7 may be provided to disable activity of an instrument temporarily. The console 2 in the illustrated system 10 may also be configurable to meet individual user preferences. For example, in the illustrated example, the pendant 8 and the touch screen 5 are shown on the left side of the console 2, but they may also be relocated to the right side of the console 2. Various numbers of display screens may be provided. Additionally or alternatively, a bedside console 3 may be provided for bedside control of the of the instrument driver 16 if desired. Further, optional keyboard may be connected to the console 2 for inputting user data. The workstation 2 may also be mounted on a set of casters or wheels to allow easy movement of the workstation 2 from one location to another, e.g., within the operating room or catheter laboratory. Further aspects of examples of suitable MID 12, and workstation 2 arrangements are described in further detail in U.S. patent application Ser. No. 11/481,433 and U.S. Provisional Patent Application No. 60/840,331, the contents of which were previously incorporated herein by reference.

As shown in FIG. 1, the support assembly 20 is configured for supporting or carrying the instrument driver 16 over the operating table 22. One suitable support assembly 20 has an arcuate shape and is configured to position the instrument driver 16 above a patient lying on the table 22. The support assembly 20 may be configured to movably support the instrument driver 16 and to allow convenient access to a desired location relative to the patient. The support assembly 20 may also be configured to lock the instrument driver 16 into a certain position.

In the illustrated example, the support assembly 20 is mounted to an edge of the operating table 22 such that a catheter and sheath instruments 18, 30 mounted on the instrument driver 16 can be positioned for insertion into a patient. The instrument driver 16 is controllable to maneuver the catheter and/or sheath instruments 18, 30 within the patient during a surgical procedure. The distal portion of the setup joint 20 also includes a control lever 33 for maneuvering the setup joint 20. Although the figures illustrate a single guide catheter 18 and sheath assembly 30 mounted on a single instrument driver 16, embodiments may be implemented in systems 10 having other configurations. For example, embodiments may be implemented in systems 10 that include a plurality of instrument drivers 16 on which a plurality of catheter/sheath instruments 18, 30 can be controlled. Further aspects of a suitable support assembly 20 are described in U.S. patent application Ser. No. 11/481,433 and U.S. Provisional Patent Application No. 60/879,911, the contents of which are expressly incorporated herein by reference. Referring to FIG. 2, the support assembly 20 may be mounted to an operating table 22 using a universal adapter base plate assembly 39, similar to those described in detail in U.S. Provisional Patent Application No. 60/899,048, incorporated by reference herein in its entirety. The adapter plate assembly 39 mounts directly to the operating table 22 using clamp assemblies, and the support assembly 20 may be mounted to the adapter plate assembly 39. One suitable adapter plate assembly 39 includes two large, flat main plates which are positioned on top of the operating table 22. The assembly 39 provides for various adjustments to allow it to be mounted to different types of operating tables 22. An edge of the adapter plate assembly 39 may include a rail that mimics the construction of a traditional surgical bedrail. By placing this rail on the adapter plate itself, a user may be assured that the component dimensions provide for proper mounting of the support assembly 20. Furthermore, the large, flat surface of the main plate provides stability by distributing the weight of the support assembly 20 and instrument driver 16 over an area of the table 22, whereas a support assembly 20 mounted directly to the operating table 22 rail may cause its entire load to be placed on a limited and less supportive section of the table 22. Additionally or alternatively, a bedside rail 13 may be provided which may couple the support assembly 20 to the operating table 22. The bedside rail may include a leadscrew mechanism which will enable the support assembly to translate linearly along the edge of the bed, resulting in a translation of the instrument driver 16 and ultimately a translation in the insert direction of the catheter and sheath instruments 18/30.

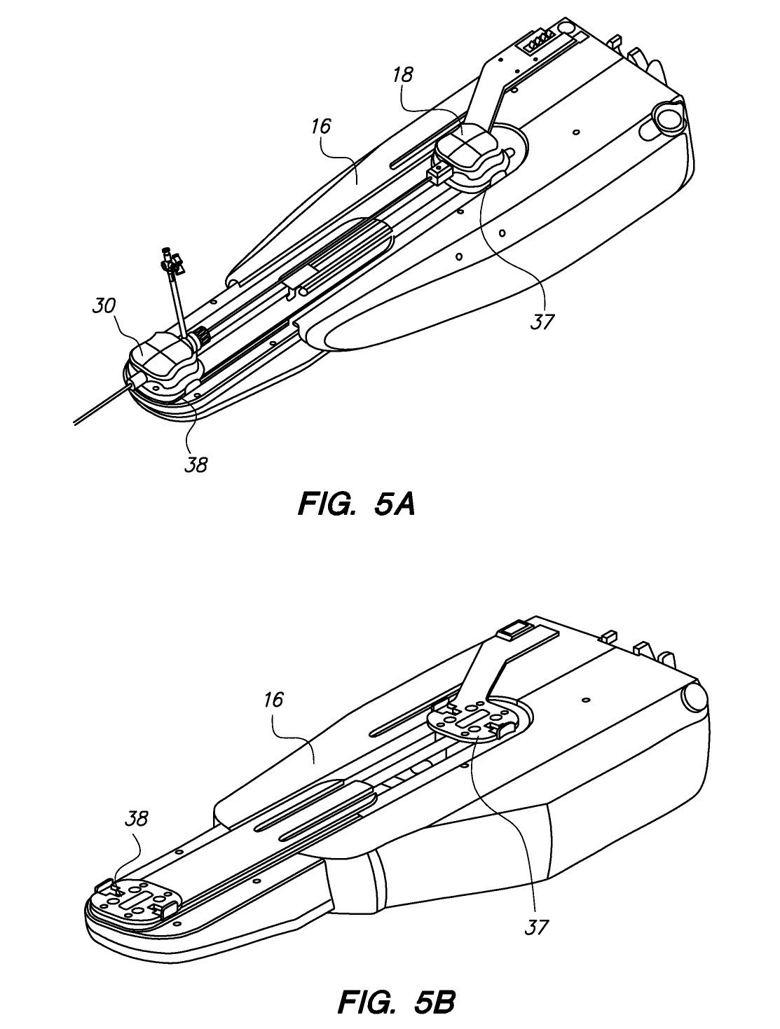

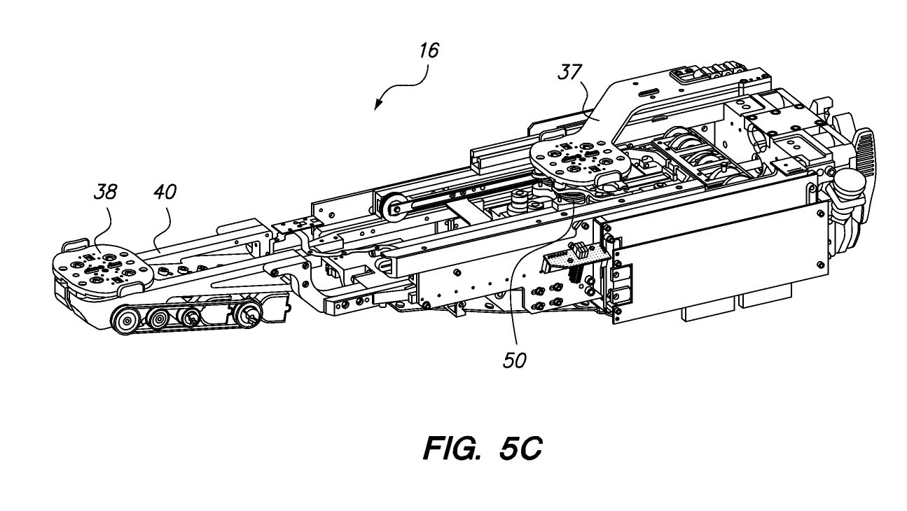

FIGS. 5A-C illustrate the instrument drive 16 with various components installed. FIG. 5A illustrates the instrument driver 16 with the instrument assembly 11 installed including the sheath instrument 30 and the associated guide or catheter instrument 18 while FIG. 5B illustrates the instrument driver 16 without an attached instrument assembly 11. The sheath instrument 30 and the associated guide instrument 18 are mounted to associated mounting plates 37, 38 on a top portion of the instrument driver 16. FIG. 5C illustrates the instrument driver 16 with skins removed to illustrate internal components. Embodiments described are similar to those described in detail in U.S. patent application Ser. Nos. 11/678,001, 11/678,016, and 11/804,585, each incorporated by reference herein in its entirety.

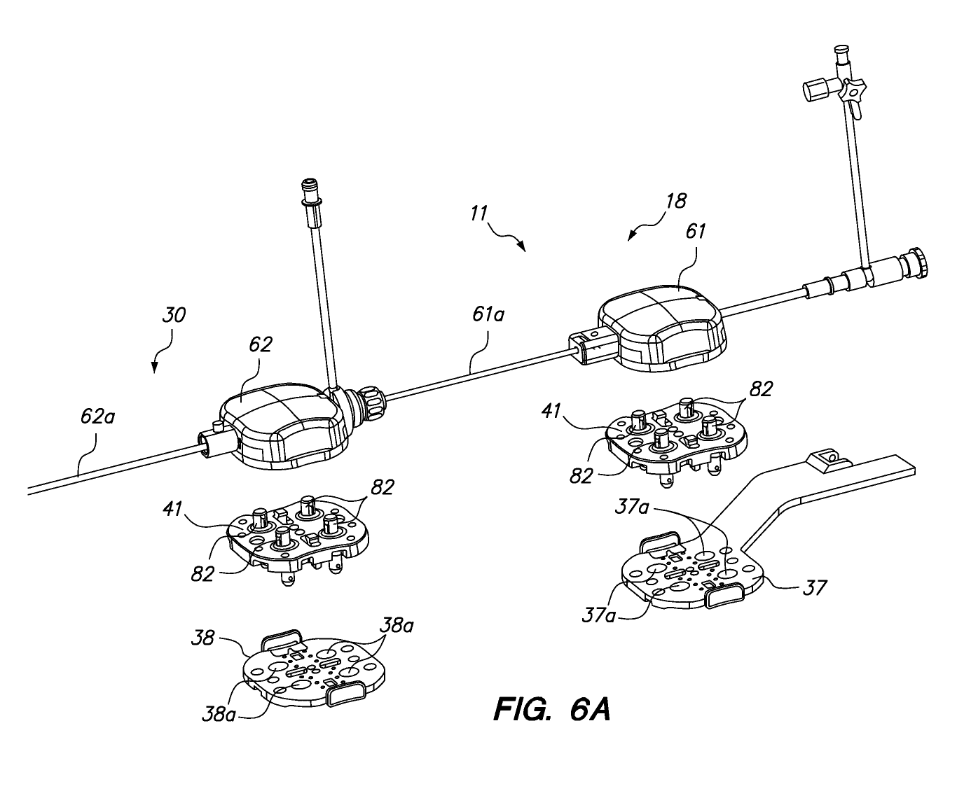

Referring to FIGS. 6A-B, the assembly 11 that includes the sheath instrument 30 and the guide or catheter instrument 18 positioned over their respective mounting plates 38, 37 is illustrated removed from the instrument driver 16. Additionally a sterile adaptor 41 can be used to couple each of the sheath and guide instruments to their respective mounting plates. The catheter instrument 18 includes a guide catheter instrument member 61a, and the sheath instrument 30 includes a sheath instrument member 62a. The guide catheter instrument member 61a is coaxially interfaced with the sheath instrument member 62a by inserting the guide catheter instrument member 61a into a working lumen of the sheath catheter member 62a. As shown in FIG. 6A, the sheath instrument 30 and the guide or catheter instrument 18 are coaxially disposed for mounting onto the instrument driver 16. However, it should be understood that the sheath instrument 16 may be used without a guide or catheter instrument 18, or the guide or catheter instrument 18 may be used without a sheath instrument 30. In such cases, the sheath instrument 16 or the catheter instrument 18 may be mounted onto the instrument driver 16 individually. With the coaxial arrangement as shown in FIG. 6A, a guide catheter splayer 61 is located proximally relative to, or behind, a sheath splayer 62 such that the guide catheter member 61a can be inserted into and removed from the sheath catheter member 61b.

The splayers 61, 62 are configured to steer the members 61a, 61b, respectively. In the illustrated embodiments, each of the splayers 61, 62 includes drivable elements therein configured to apply tension to different respective wires inside the member 61a/61b to thereby steer the distal end of the member 61a/61b. In some embodiments, the drivable elements may be actuated in response to a control signal from a controller, which receives an input signal from the work station 2, and generates the control signal in response to the input signal. Also, in the illustrated embodiments, the splayers 61, 62 may be translated relative to the instrument driver 16. In some embodiments, the instrument driver 16 may be configured to advance and retract each of the splayers 61, 62, so that the catheter instrument 18 and the sheath instrument 30 may be advanced distally and retracted proximally.

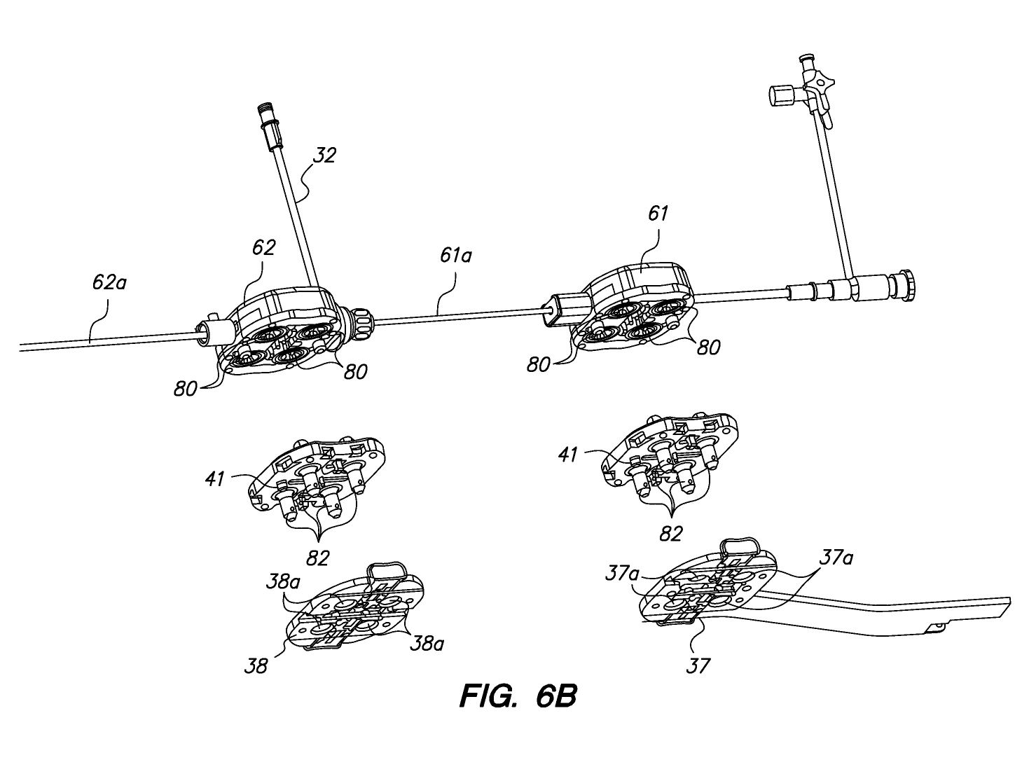

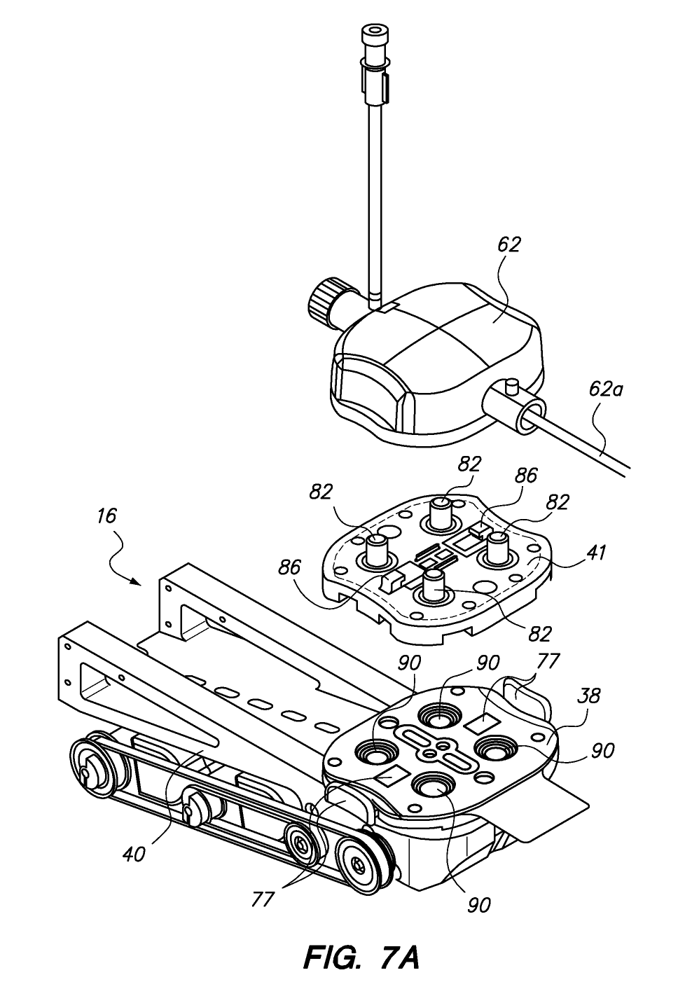

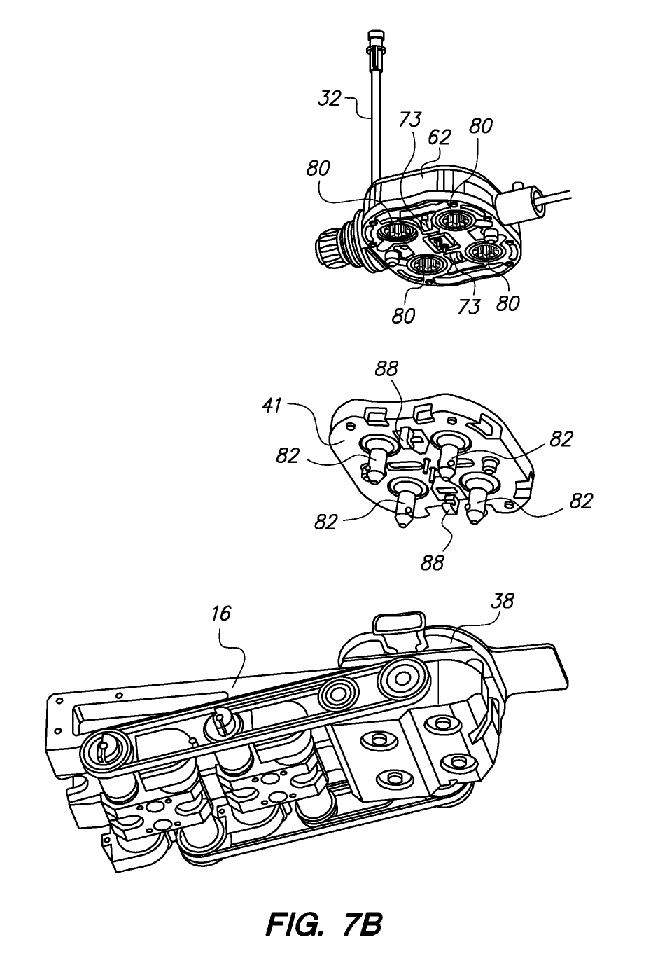

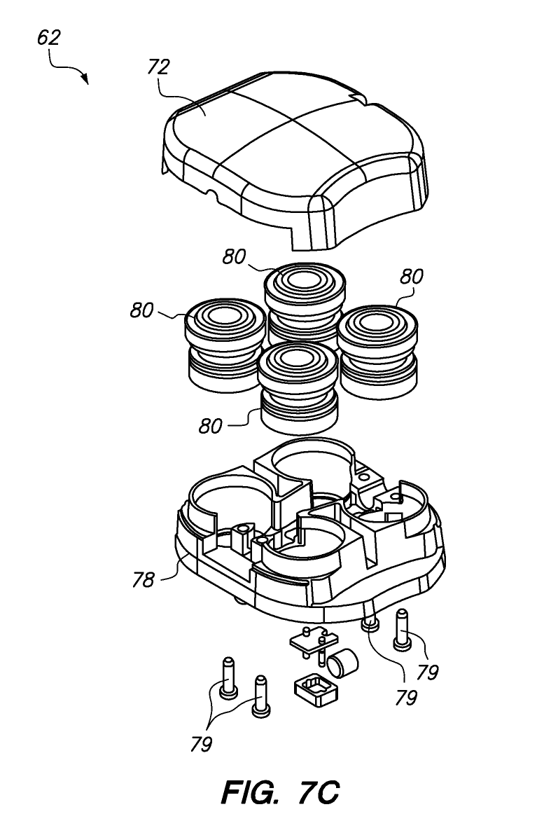

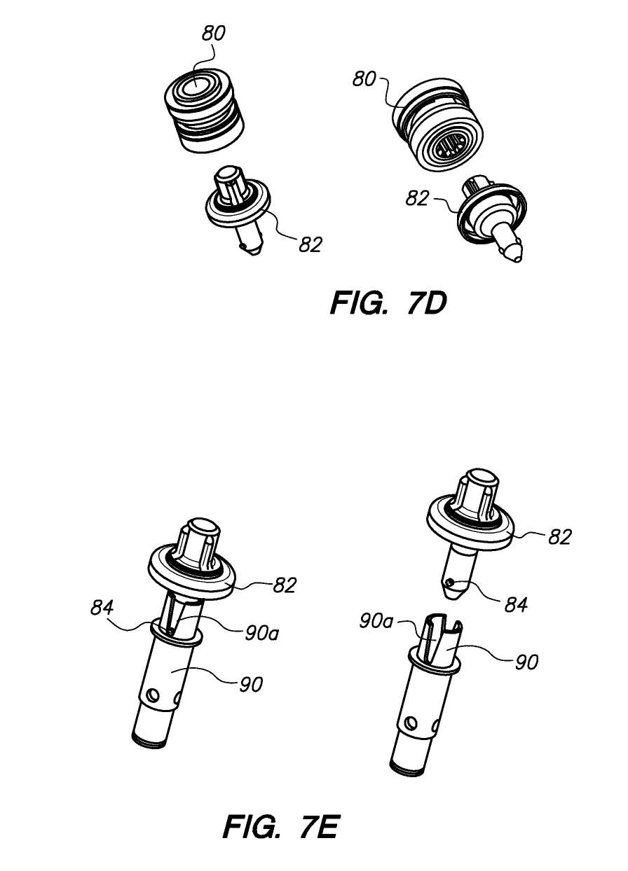

FIGS. 7A and 7B illustrate the sheath splayer 62 of one embodiment illustrated with the sterile adaptor 41 and mounting plate 38 coupled to a portion of the instrument driver shown with only a set of actuation mechanisms that will be described later in detail. As shown in FIG. 6A, the sheath and guide splayers 62, 61, appear similar physically in construction with the exception of differences in a valve purge tube 32. It should be noted that the purge tube 32 may or may not be included for either the guide or sheath splayer. The sheath splayer 62 will be described herein. However it should be understood that the guide splayer 61 is of similar construction, and components of the sheath splayer 62 can be repeated for the guide splayer 61.

As illustrated in FIG. 7C, the splayer 62 includes a splayer cover 72 fixably coupled to a splayer base assembly 78 using four screws 79. The splayer base 78 having four cavities to receive and house pulley assemblies 80 is used for both the guide splayer 61 and sheath splayer 62. For this embodiment of a sheath splayer 62, four cavities of the splayer base 78 are populated with pulley assemblies 80 but it should be understood that varying numbers of cavities may be populated leaving remaining cavities open. The guide splayer 61 may have all its cavities populated with four pulley assemblies 80 for pulling four respective wires, as can be seen in FIG. 6B. The splayer base 78 of this implementation can be constructed from injection molded polycarbonate.

During splayer 62 assembly, the pulley assembly 80 is put together and mated with a catheter pull wire or control element (not shown). The pull wire (not shown) runs down the length of a catheter from distal to proximal end then is wound about the pulley. By rotating the pulley, the pull wire bends the distal tip of the catheter controlling its bend.

Referring back to FIGS. 6A-6B, when a catheter is prepared for use with an instrument, its splayer is mounted onto its appropriate mounting plate via a sterile adaptor. In this case, the sheath splayer 62 is placed onto the sheath mounting plate 38 and the guide splayer 61 is place onto the guide mounting plate 37 via sterile adaptors 41. Referring to FIG. 7A-B, the pulley assemblies 80 are configured to couple to floating shafts 82 on the splayer adaptor 41 which in turn are configured to couple to sleeve receptacles 90. In the illustrated example, each mounting plates 37, 38 has four openings 37a, 38a that are designed to receive the corresponding floating shafts 84 attached to and extending from the sterile adaptors 41 coupled to the splayers 61, 62. In the example illustrated in FIG. 6B, four floating shafts 82 of the sterile adaptor 41 are insertable within the openings 38a of the sheath mounting plate 38 as the splayer 62 is mounted onto the RCM. Similarly, four floating shafts 82 of the sterile adaptor 41 are insertable within the four apertures or openings 37a of the guide interface plate 37. Referring to FIGS. 7D-E, the coupling of the pulley assemblies 80 to floating shafts 82 and floating shafts 82 to sleeve receptacles 90 is illustrated. FIG. 7D illustrates top and bottom perspective views of the pulley assembly 80 positioned above the floating shaft 82 where the bottom of the pulley assembly 80 is configured to mate with splines on the top of the floating shaft 82. FIG. 7E illustrates the floating shaft 82 installed and un-installed onto the sleeve receptacle 90. The sleeve receptacles can include a notch 90a shaped to accept a pin 84 on the floating shaft 82.

Referring back to FIGS. 7A-B, the sheath splayer 62 is shown having latches 73 which may couple to hooks 86. By depressing the latches 73, the splayer 62 may be locked and unlocked to the sterile adaptor 41. The sterile adaptor in turn is configured having mounting hooks 88 which couple to sliding latches 77 on the mounting plate 83. The sliding latches 77 can be spring loaded to allow the adaptor plate 41 to be locked to the mounting plate 38 by applying downward force on the adaptor plate 41. The sliding latches can be depressed to release the adaptor plate 41 when desired.

The sheath interface mounting plate 38 as illustrated in FIGS. 6A and 6B is similar to the guide interface mounting plate 37, and thus, similar details are not repeated. One difference between the plates 37, 38 may be the shape of the plates. For example, the guide interface plate 37 includes a narrow, elongated segment, which may be used with, for example, a dither mechanism or the elongate member manipulator 24. Both plates 37, 38 include a plurality of openings 37a, 38a to receive floating shafts 82 and latches 73 from sterile adaptors 41. The splayers 61/62, sterile adaptors 41, and mounting plates 37/38 are all described in greater detail in U.S. patent application Ser. No. 13/173,994, filed on Jun. 30, 2011, the entire disclosure of which is expressly incorporated by reference herein.

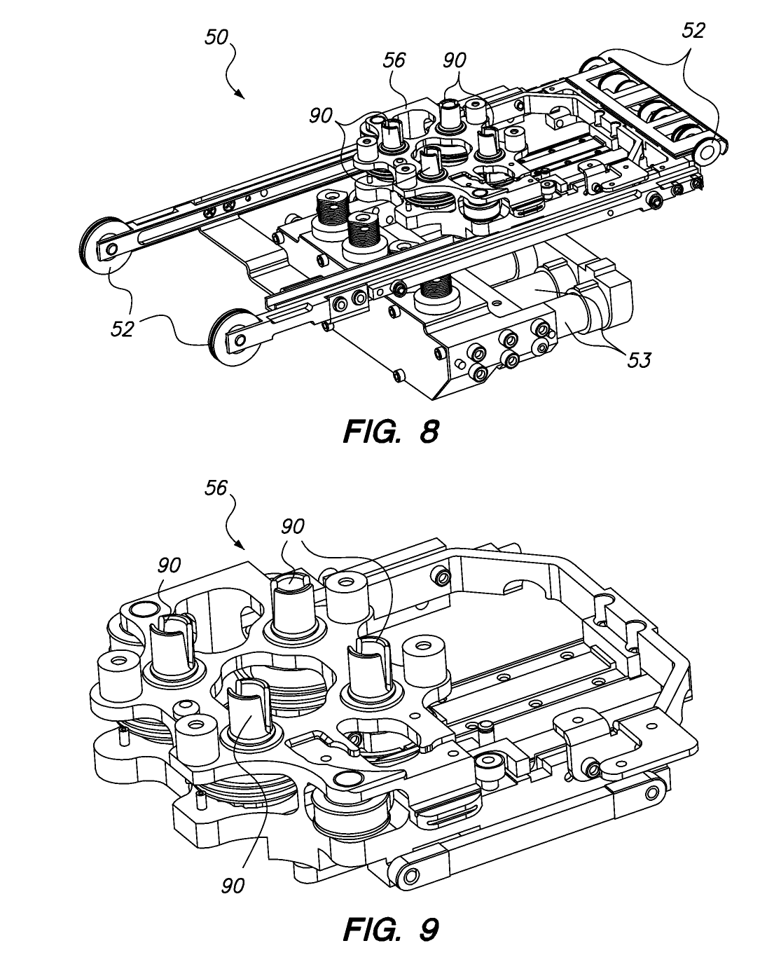

Referring back to FIG. 5C the instrument driver 16 is illustrated with mounting plates 37,38 fixably coupled to a guide carriage 50, and a sheath drive block 40, respectively. FIG. 8 illustrates the guide carriage 50 removed from the instrument driver 16 coupled to cabling (not shown) and associated guide motors 53. The guide carriage 50 includes a funicular assembly 56 which is illustrated in FIG. 9. The funicular assembly 56 includes the four sleeve receptacles 90. As previously described, the floating shafts 82 of the sterile adaptor 41 first insert through the openings 37a in the mounting plate 37. They then engage with the sleeve receptacles 90

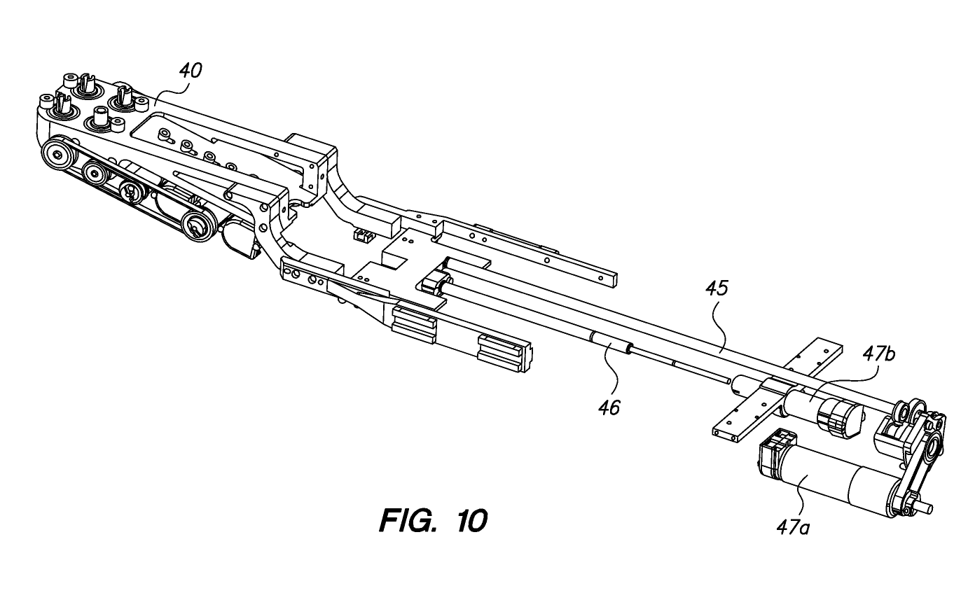

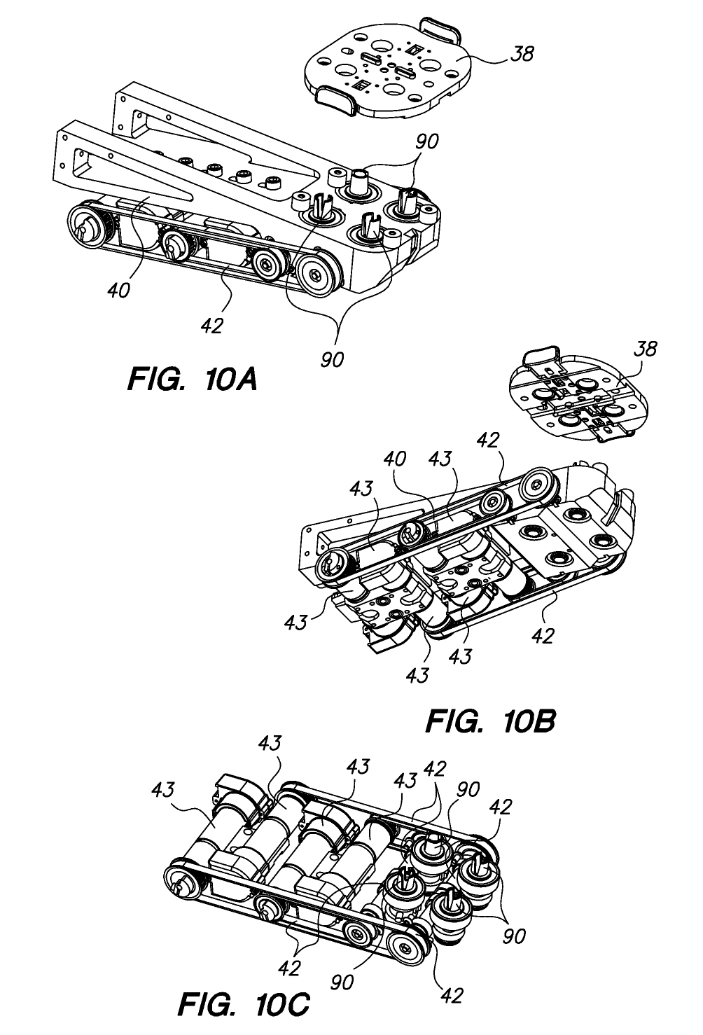

Referring back to FIG. 8, a set of cables (not shown) wound around a set of pulleys 52, are coupled on one end to a set of guide motors 53 and the other end to the sleeve receptacles 90. Note that only two of four motors can be seen in FIG. 8. The drive motors 53 are actuated to rotationally drive the sleeves 90. The catheter assembly 18 with its splayer 61 mounted onto the instrument drive 16 would have its pulley assemblies 80 coupled to corresponding sleeves 90 via floating shafts 82. As the sleeves 90 are rotated, the pins 84 of the floating shafts 82 are seated in the V-shaped notches and are engaged by the rotating sleeves 90, thus causing the floating shafts 82 and associated pulley assemblies 80 to also rotate. The pulley assemblies 80 in turn cause the control elements (e.g., wires) coupled thereto to manipulate the distal tip of the catheter instrument 30 member in response thereto. FIGS. 10A and 10B illustrate top and bottom perspective views of the sheath output plate 38 exploded from the sheath block 40 and motor driven interfaces 42 which are coupled to sheath articulation motors 43. FIG. 10C illustrates sheath articulation motors 43 coupled to the motor driven interfaces 42 which includes a set of belts, shafts, and gears which drive receptacle sleeves 90 (which are similar in construction and functionality to the receptacle sleeves previously described for the guide funicular assembly). When the sheath splayer pulley assemblies 80 and sterile adaptor floating shafts 82 are coupled to the receptacle sleeves 90, the sheath articulation motors 43 drive the receptacle sleeves 90 causing the sheath instrument 30 to bend in the same manner described for the guide instrument.

During use, the catheter instrument 18 is inserted within a central lumen of the sheath instrument 30 such that the instruments 18, 30 are arranged in a coaxial manner as previously described. Although the instruments 18, 30 are arranged coaxially, movement of each instrument 18, 30 can be controlled and manipulated independently. For this purpose, motors within the instrument driver 16 are controlled such that the drive and sheath carriages coupled to the mounting plates 37, 38 are driven forwards and backwards independently on linear bearings each with leadscrew actuation. FIG. 10 illustrates the sheath drive block 40 removed from the instrument driver coupled to two independently-actuated lead screw 45, 46 mechanisms driven by guide and sheath insert motors 47a,47b. Note the guide carriage is not shown. In the illustrated embodiment, the sheath insertion motor 47b is coupled to a sheath insert leadscrew 46 that is designed to move the sheath articulation assembly forwards and backwards, thus sliding a mounted sheath catheter instrument (not shown) forwards and backwards. The insert motion of the guide carriage can be actuated with a similar motorized leadscrew actuation where a guide insert motor 47a is coupled to the guide insert leadscrew 45 via a belt.

Referring back to FIGS. 1, 4 and 6A, in order to accurately steer the robotic sheath 62a or guide catheter 61a from an operator work station 2, a control structure may be implemented which allows a user to send commands through input devices such as the pendant 8 or MID 12 that will result in desired motion of the sheath 62a and guide 61a. In some embodiments, the sheath 62a and/or the guide 61a may each have four control wires for bending the instrument in different directions. Referring to FIGS. 11A-H, the basic kinematics of a catheter 120 with four control elements 122a, 122b, 122c, 122d is shown. The catheter 120 may be component 61a or component 62a in some embodiments. Referring to FIGS. 11A-B, as tension is placed only upon the bottom control element 122c, the catheter bends downward, as shown in FIG. 11A. Similarly, pulling the left control element 122d in FIGS. 11C-D bends the catheter left, pulling the right control element 122b in FIGS. 11E-F bends the catheter right, and pulling the top control element 122a in FIGS. 11G-H bends the catheter up. As will be apparent to those skilled in the art, well-known combinations of applied tension about the various control elements results in a variety of bending configurations at the tip of the catheter member 120.

The kinematic relationships for many catheter instrument embodiments may be modeled by applying conventional mechanics relationships. In summary, a control-element-steered catheter instrument is controlled through a set of actuated inputs. In a four-control-element catheter instrument, for example, there are two degrees of motion actuation, pitch and yaw, which both have + and − directions. Other motorized tension relationships may drive other instruments, active tensioning, or insertion or roll of the catheter instrument. The relationship between actuated inputs and the catheter's end point position as a function of the actuated inputs is referred to as the “kinematics” of the catheter.

To accurately coordinate and control actuations of various motors within an instrument driver from a remote operator control station such as that depicted in FIG. 1, a computerized control and visualization system may be employed. The control system embodiments that follow are described in reference to a particular control systems interface, namely the SimuLink™ and XPC™ control interfaces available from The Mathworks Inc., and PC-based computerized hardware configurations. However, one of ordinary skilled in the art having the benefit of this disclosure would appreciate that many other control system configurations may be utilized, which may include various pieces of specialized hardware, in place of more flexible software controls running on one or more computer systems.

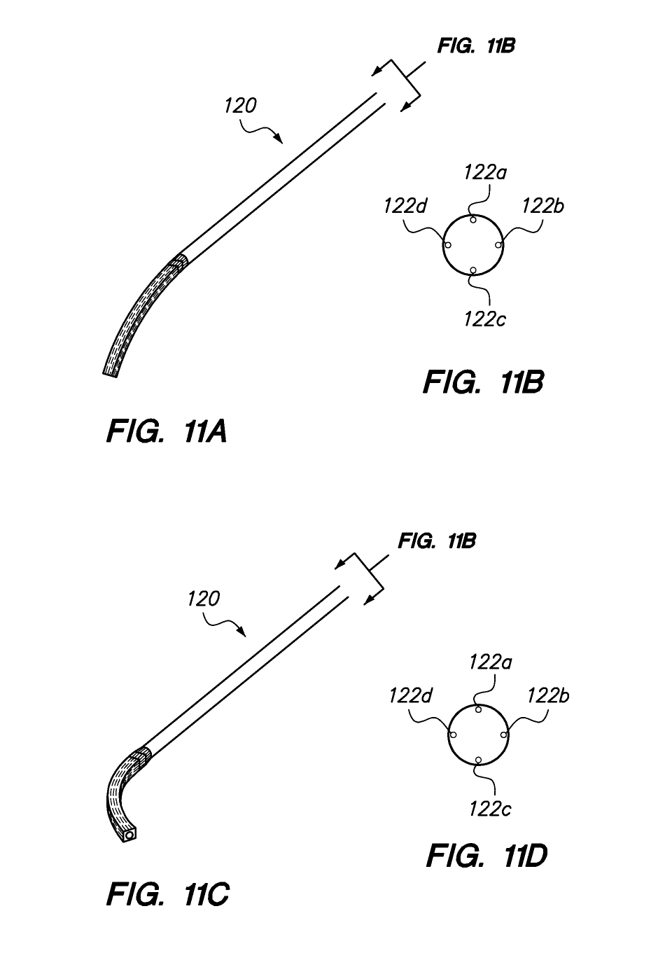

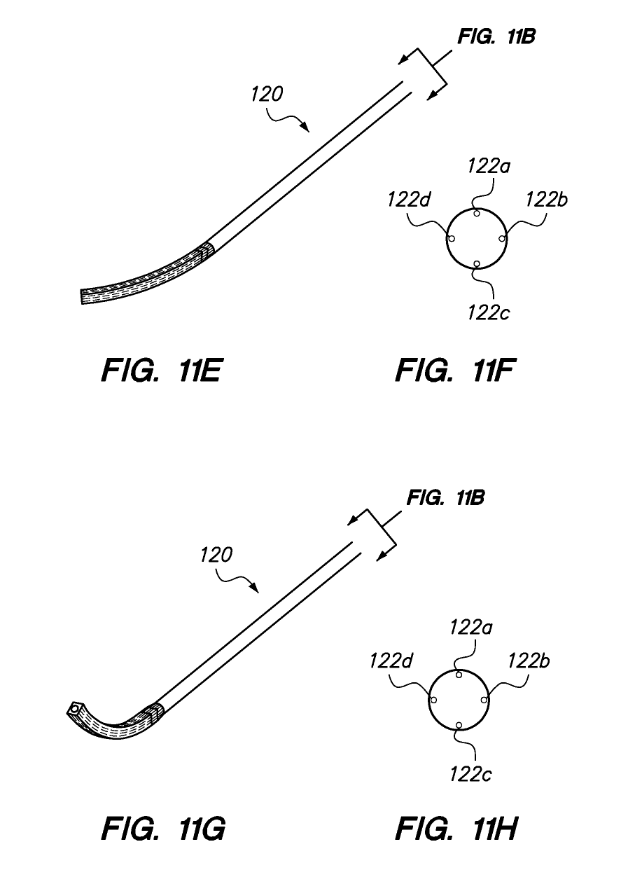

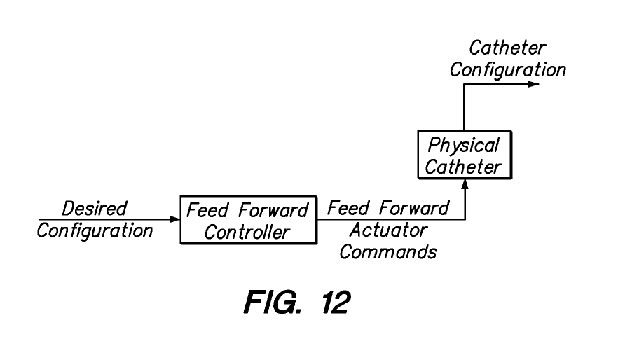

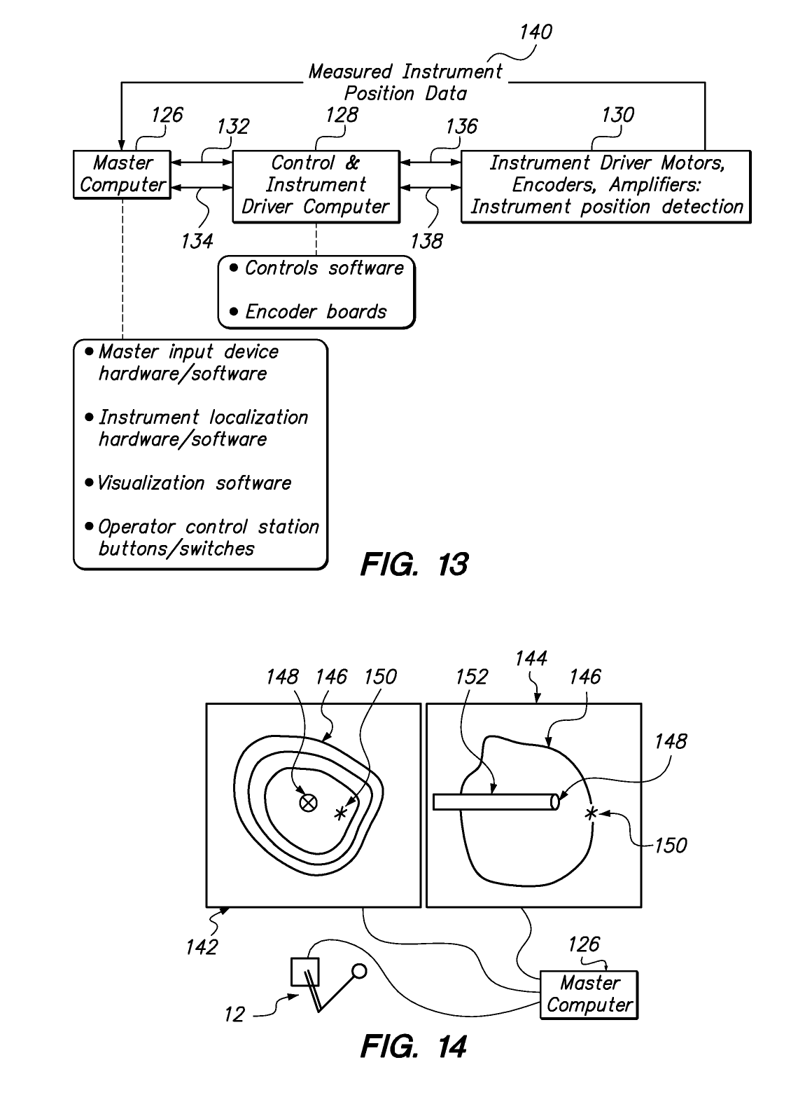

FIGS. 12-13 illustrate examples of a control structure for moving the catheter 61a and/or the sheath 62a in accordance with some embodiments. In one embodiment, the catheter (or other shapeable instrument) is controlled in an open-loop manner as shown in FIG. 12. In this type of open loop control model, the shape configuration command comes in to the beam mechanics, is translated to beam moments and forces, then is translated to tendon tensions given the actuator geometry, and finally into tendon displacement given the entire deformed geometry.

Referring to FIG. 13, an overview of other embodiment of a control system flow is depicted. A master computer 400 running master input device software, visualization software, instrument localization software, and software to interface with operator control station buttons and/or switches is depicted. In one embodiment, the master input device software is a proprietary module packaged with an off-the-shelf master input device system, such as the Phantom™ from Sensible Devices Corporation, which is configured to communicate with the Phantom™ hardware at a relatively high frequency as prescribed by the manufacturer. Other suitable master input devices, such as the master input device 12 depicted in FIG. 2 are available from suppliers such as Force Dimension of Lausanne, Switzerland. The master input device 12 may also have haptics capability to facilitate feedback to the operator, and the software modules pertinent to such functionality may also be operated on the master computer 126.

Referring to FIG. 13, in one embodiment, visualization software runs on the master computer 126 to facilitate real-time driving and navigation of one or more steerable instruments. In one embodiment, visualization software provides an operator at an operator control station, such as that depicted in FIG. 2, with a digitized “dashboard” or “windshield” display to enhance instinctive drivability of the pertinent instrumentation within the pertinent tissue structures. Referring to FIG. 14, a simple illustration is useful to explain one embodiment of a preferred relationship between visualization and navigation with a master input device 12. In the depicted embodiment, two display views 142, 144 are shown. One preferably represents a primary 142 navigation view, and one may represent a secondary 144 navigation view. To facilitate instinctive operation of the system, it is preferable to have the master input device coordinate system at least approximately synchronized with the coordinate system of at least one of the two views. Further, it is preferable to provide the operator with one or more secondary views which may be helpful in navigating through challenging tissue structure pathways and geometries.

Referring still to FIG. 14, if an operator is attempting to navigate a steerable catheter in order to, for example, contact a particular tissue location with the catheter's distal tip, a useful primary navigation view 142 may comprise a three dimensional digital model of the pertinent tissue structures 146 through which the operator is navigating the catheter with the master input device 12, along with a representation of the catheter distal tip location 148 as viewed along the longitudinal axis of the catheter near the distal tip. This embodiment illustrates a representation of a targeted tissue structure location 150, which may be desired in addition to the tissue digital model 146 information. A useful secondary view 144, displayed upon a different monitor, in a different window upon the same monitor, or within the same user interface window, for example, comprises an orthogonal view depicting the catheter tip representation 148, and also perhaps a catheter body representation 152, to facilitate the operator's driving of the catheter tip toward the desired targeted tissue location 150.

In one embodiment, subsequent to development and display of a digital model of pertinent tissue structures, an operator may select one primary and at least one secondary view to facilitate navigation of the instrumentation. By selecting which view is a primary view, the user can automatically toggle a master input device 12 coordinate system to synchronize with the selected primary view. In an embodiment with the leftmost depicted view 142 selected as the primary view, to navigate toward the targeted tissue site 150, the operator should manipulate the master input device 12 forward, to the right, and down. The right view will provide valued navigation information, but will not be as instinctive from a “driving” perspective.

To illustrate: if the operator wishes to insert the catheter tip toward the targeted tissue site 150 watching only the rightmost view 144 without the master input device 12 coordinate system synchronized with such view, the operator would have to remember that pushing straight ahead on the master input device will make the distal tip representation 148 move to the right on the rightmost display 144. Should the operator decide to toggle the system to use the rightmost view 144 as the primary navigation view, the coordinate system of the master input device 12 is then synchronized with that of the rightmost view 144, enabling the operator to move the catheter tip 148 closer to the desired targeted tissue location 150 by manipulating the master input device 12 down and to the right. The synchronization of coordinate systems may be conducted using fairly conventional mathematic relationships which are described in detail in the aforementioned applications incorporated by reference.

Referring back to embodiment of FIG. 13, the master computer 126 also comprises software and hardware interfaces to operator control station buttons, switches, and other input devices which may be utilized, for example, to “freeze” the system by functionally disengaging the master input device as a controls input, or provide toggling between various scaling ratios desired by the operator for manipulated inputs at the master input device 12. The master computer 126 has two separate functional connections with the control and instrument driver computer 128: one connection 132 for passing controls and visualization related commands, such as desired XYZ (in the catheter coordinate system) commands, and one connection 134 for passing safety signal commands. Similarly, the control and instrument driver computer 128 has two separate functional connections with the instrument and instrument driver hardware 130: one connection 136 for passing control and visualization related commands such as required-torque-related voltages to the amplifiers to drive the motors and encoders, and one connection 138 for passing safety signal commands. Also shown in the signal flow overview of FIG. 13 is a pathway 140 between the physical instrument and instrument driver hardware 130 back to the master computer 126 to depict a closed loop system embodiment wherein instrument localization technology is utilized to determine the actual position of the instrument to minimize navigation and control error.

II. Elongate Member

Referring to FIG. 15, the elongate member 26 of FIG. 3 will now be described in further detail. As shown in FIG. 15, the elongate member 26 has a distal end 300, a proximal end 302, and a body 304 extending between the distal end 300 and the proximal end 302. The distal end 300 of the elongate member 26 includes a port 310 (at the distal tip) for delivering fluid at a target location. The elongate member 26 also includes a plurality of side ports 312 (e.g., eight side ports 312) located along a length of the elongate member 26 for delivering fluid to the target location. In other embodiments, the elongate member 26 may not include the plurality of ports 312 or the distal port 310. The elongate member 26 also has a sharp distal tip configured to pierce tissue (e.g., patient's skin, tissue at target site, etc.). In some embodiments, the distal end 300 of the elongate member 26 with the sharp tip may be implemented as a needle. In other embodiments, the tip of the elongate member 26 may be blunt. The elongate member 26 also includes a flexible section 320 that is proximal to the plurality of ports 312. The flexible section 320 is more flexible than the section 322 that is distal to the section 320. In some embodiments, the flexible section 320 may be created by providing one or more openings 324 through a wall of the body 304 to decrease the bending stiffness at the section 320. For example, the opening(s) may be cutout(s). In some embodiments, the cutout may have a spiral configuration. In other embodiments, the cutout may have other configurations. The elongate member 26 further includes a jacket 330 disposed over at least a portion of the body 304 for covering the opening(s) 324 so that fluid being delivered by the elongate member 26 is contained therein. The jacket 330 may also be used to electrically insulate the portion of the elongate member 26 that is proximal to the section 322. In other embodiments, instead of providing opening(s) at the body 304 to create the flexible section 320, the flexible section 320 may be created using a wire mesh, a cage structure, a micro spine, etc., which is attached to the distal section 322.

The elongate member 26 may be made from a variety of materials. In some embodiments, the elongate member 26 may be made from Nitinol. In other embodiments, the elongate member 26 may be made from other metals or alloys. In the illustrated embodiments, the flexible section 320 is configured to allow the elongate member 26 to have sufficient bending flexibility so that the elongate member 26 may be bent easily while inside a patient's body. The flexible section 320 is also configured to allow the elongate member 26 to have sufficient axial stiffness so that the distal tip of the elongate member 26 may be used to pierce tissue in response to an axial force applied along a longitudinal axis of the elongate member 26. In some embodiments, the flexible section 320 is located close to the distal end 300 of the elongate member 26, and the length of the flexible section 320 is less than 4 inches, and more preferably, less than 2 inches (e.g., 1 inch or less). In other embodiments, the flexible section 320 may extend along a majority of the length of the elongate member 26. For example, in some embodiments, the flexible section 320 may extend proximally to the proximal end 302 of the elongate member 26.

As shown in the illustrated embodiments, the elongate member 26 is disposed within a lumen of the catheter 61a during use. The proximal end 302 of the elongate member 26 exits from the catheter splayer 61, and is coupled to the elongate member manipulator 24. The elongate member manipulator 24 is configured to move the elongate member 26 during an operation. In the illustrated embodiments, the proximal end 302 of the elongate member 26 is electrically coupled to a RF generator 350, which provides a current to the distal end 300 of the elongate member 26 during use. A return electrode 352 may also be coupled to the RF generator 350. During an operation, the return electrode 352 is placed on a patient's skin, and the distal end 300 of the elongate member is inserted into the patient and is placed at a target location (e.g., at tissue desired to be ablated). The RF generator 350 is then activated to deliver current to the distal end 300 of the elongate member 26. The current flow from the distal end 300 to tissue inside the patient, and the return electrode 352 completes the current path, thereby allowing the distal end 300 to ablate the target tissue through radiofrequency ablation.

In some embodiments, the elongate member 26 may be 140 mm long with an outer diameter of 0.035 inch. In other embodiments, the elongate member 26 may have other lengths and outer diameter of other values. Also, in some embodiments, the distal 8 cm of the elongate member 26 may have an outer diameter of 0.025 inch with an inner diameter of 0.018 inch, and may be made from Nitinol. The proximal shaft may include a stainless steel hypotube having an inner diameter of 0.026 inch with an outer diameter of 0.033 inch, wherein the hypotube may be insulated with a 0.001 inch thick polyimide. In other embodiments, the elongate member 26 may have different configurations (e.g., may be made from different material, and/or may have other dimensions) from the examples described above. An electrical conductor may be coupled to the hypotube, and connected to a terminal of the RF generator 350.

In the illustrated embodiments, the proximal end 302 of the elongate member 26 is also coupled to a material source 360. In one implementation, a male luer fitting may be attached to the proximal end of the elongate member 26, and the luer is then connected to a peristaltic pump (an example of the material source 360). The pump may provide a flow rate of 2-4 mL/minute. In other embodiments, the pump may provide other flow rates. In some embodiments, the material source 360 may be in fluid communication with internal lumen in the body 304 of the elongate member 26. Also, in some embodiments, the material source 360 may contain fluid, such as an agent, a drug (e.g., chemotherapy drug), saline, cooling fluid (e.g., saline), or any of other types of fluid. In one method of use, while the distal end 300 of the elongate member 26 is delivering energy to treat tissue, cooling fluid may be delivered from the source 360 to the target site to thereby control a manner in which the energy is being delivered to the tissue. In some cases, by delivering fluid at the target site during tissue ablation, the tissue may be ablated in a more controlled or desirable (e.g., gradual) manner, thereby allowing a larger lesion to be created by the ablation process. In particular, the cooling fluid may increase the effective thermal mass so that more energy may be delivered deeper into the target tissue. Without irrigation, local necrosis around the elongate member 26 may increase local impedance, and RF energy may stop penetrating tissue at a relatively low power setting. With irrigation, higher power setting may be applied, and the necrotic lesion created by the elongate member 26 may exceed 3 cm in cross section. In other embodiments, the material source 360 may contain embolic material configured to occlude a vessel. In further embodiments, the material source 360 may contain other substances, such as radioactive seeds, a composition that causes tissue reaction, or a composition that causes tissue injury, etc. In still further embodiments, the lumen inside the elongate member 26 may be used to house another device, such as an optical fiber. The optical fiber may be used to image tissue inside the patient as the elongate member 26 is being positioned inside the patient. When the elongate member 26 is desirably positioned, the optical fiber may be removed from the lumen of the elongate member 26, and the lumen may then be used to deliver a substance to a target site.

In other embodiments, instead of using an electrode that is placed outside the patient, the elongate member 26 may include a plurality of electrodes for providing radiofrequency energy in a bi-polar configuration. For example, as shown in FIG. 16, in other embodiments, the distal end 300 of the elongate member 26 may include two electrodes 370a, 370b. The electrodes 370a, 370b are electrically insulated from each other along the length of the elongate member 26. The electrodes 370a, 370b are electrically coupled to respective terminals at the RF generator 350. The electrical insulation of the electrodes 370a, 370b may be achieved by providing electrically insulative material (e.g., polymer, plastic, etc.) along the length of the elongate member 26 that separates the electrodes 370a, 370b.

In some embodiments, the elongate member 26 may optionally further include one or more radio opaque markers (e.g., a radio opaque band) located at the distal end 300 or anywhere along the length of the elongate member 26. The marker(s) allows the elongate member 26 to be visualized using an imaging technique during a procedure. In other embodiments, the elongate member 26 may include one or more localization coils, or one or more transmitters for transmitting localization signals, at the distal end 300 or anywhere along the length of the elongate member 26, for allowing a three dimensional coordinate of the elongate member 26 to be determined. In further embodiments, the elongate member 26 may include a fiber (e.g., optical fiber) for localization. For example, the fiber may be disposed in a lumen in the elongate member 26, or may be embedded in a wall of the elongate member 26.

Various types of optical fibers may be used with elongate members 26 for localization. For example, a fiber optic Bragg sensing fiber may be placed inside the lumen of the elongate member 26 to sense position, shape and temperature. By applying the Bragg equation (wavelength=2*d*sin(theta)) to detect wavelength changes in reflected light, elongation in a diffraction grating pattern positioned longitudinally along a fiber or other elongate structure maybe be determined. Further, with knowledge of thermal expansion properties of fibers or other structures which carry a diffraction grating pattern, temperature readings at the site of the diffraction grating may be calculated. “Fiberoptic Bragg grating” (“FBG”) sensors or components thereof, available from suppliers such as Luna Innovations, Inc., of Blacksburg, Va., Micron Optics, Inc., of Atlanta, Ga., LxSix Photonics, Inc., of Quebec, Canada, and Ibsen Photonics AIS, of Denmark, have been used in various applications to measure strain in structures such as highway bridges and aircraft wings, and temperatures in structures such as supply cabinets.

Techniques for determining a geometric configuration of an elongated member using light transmitted through a fiber optic as well as the use of such technology in shapeable instruments have been describe in U.S. patent applications previously incorporated by reference.

In an alternative variation, a single mode optical fiber is drawn with slight imperfections that result in index of refraction variations along the fiber core. These variations result in a small amount of backscatter that is called Rayleigh scatter. Changes in strain or temperature of the optical fiber cause changes to the effective length of the optical fiber. This change in the effective length results in variation or change of the spatial position of the Rayleigh scatter points. Cross correlation techniques can measure this change in the Rayleigh scattering and can extract information regarding the strain. These techniques can include using optical frequency domain reflectometer techniques in a manner that is very similar to that associated with low reflectivity fiber gratings. A more complete discussion of these methods can be found in M. Froggatt and J. Moore, “High-spatial-resolution distributed strain measurement in optical fiber with Rayleigh scatter”, Applied Optics, Vol. 37, p. 1735, 1998 the entirety of which is incorporated by reference herein.

Methods and devices for calculating birefringence in an optical fiber based on Rayleigh scatter as well as apparatus and methods for measuring strain in an optical fiber using the spectral shift of Rayleigh scatter can be found in PCT Publication No. W02006099056 filed on Mar. 9, 2006 and U.S. Pat. No. 6,545,760 filed on Mar. 24, 2000 both of which are incorporated by reference herein. Birefringence can be used to measure axial strain and/or temperature in a waveguide. Using Rayleigh scatter to determine birefringence rather than Bragg gratings offers several advantages. First, the cost of using Rayleigh scatter measurement is less than when using Bragg gratings. Rayleigh scatter measurement permits birefringence measurements at every location in the fiber, not just at predetermined locations. Since Bragg gratings require insertion at specific measurement points along a fiber, measurement of Rayleigh scatter allows for many more measurement points. Also, the process of physically “writing” a Bragg grating into an optical fiber can be time consuming as well as compromises the strength and integrity of the fiber. Such drawbacks do not occur when using Rayleigh scatter measurement.

Also, in some embodiments, the elongate member 26 may optionally further include one or more temperature sensors (e.g., thermocouple(s)) located at the distal end 300 of the elongate member 26. During use, the temperature sensor(s) may be used to sense temperature at the distal end 300 of the elongate member 26. The sensed temperature may be transmitted to the RF generator 350, which may adjust the energy delivery based on the sensed temperature.

Although the elongate member 26 has been described as being configured to deliver radiofrequency energy for ablation, in other embodiments, the elongate member 26 may be configured to deliver other types of energy. For example, in other embodiments, the elongate member 26 may be configured to deliver ultrasound energy for tissue ablation. In such cases, the distal end 300 of the elongate member 26 may carry an ultrasound transducer configured to deliver ultrasound energy having a level that is sufficient to treat tissue. In other embodiments, the elongate member 26 may be configured to provide heat, light, radiation, or any of other types of energy, for treating tissue at a target site. Also, in other embodiments, the elongate member 26 may not have any substance delivery capability. In such cases, the elongate member 26 may not include any internal lumen, and may have a solid cross section instead. Furthermore, although the elongate member 26 is shown as being used with the catheter 61a and sheath 62a, in other embodiments, the elongate member 26 may be used with the catheter 61a without the sheath 62a.

III. Elongate Member Manipulator

The elongate member manipulator 24 of FIG. 1 will now be described in detail. In the illustrated embodiments, the manipulator 24 is configured to advance the elongate member 26 distally and proximally. In some embodiments, the manipulator 24 may advance the elongate member 26 distally relative to the catheter 61a and/or the sheath 62a. In other embodiments, the manipulator 24 may retract the elongate member 26 proximally relative to the catheter 61a and/or the sheath 62a. In further embodiments, the manipulator 24 may be configured to move the elongate member 26 in synchronization with a movement of the catheter 61a and/or the sheath 62a. For example, as the catheter 61a is being moved (e.g., advanced distally or retracted proximally) by the robotic system 10, the manipulator 24 of the system 10 also moves the elongate member 26 so that the elongate member 26 and the catheter 61a moves together, and the relative position between the elongate member 26 and the catheter 61a stays the same during the movement. Similarly, in another example, as the sheath 62a is being moved (e.g., advanced distally or retracted proximally) by the robotic system 10, the manipulator 24 of the system 10 also moves the elongate member 26 so that the elongate member 26 and the sheath 62a moves together, and the relative position between the elongate member 26 and the sheath 62a stays the same during the movement.

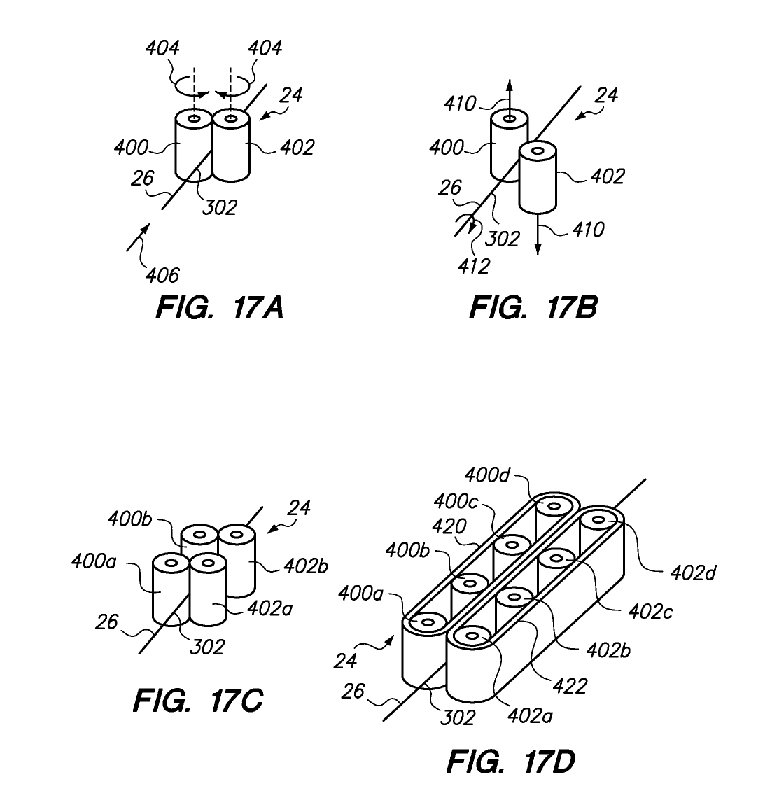

Various techniques may be employed to implement the elongate member manipulator 24. As shown in FIG. 17A, in some embodiments, the elongate member manipulator 24 may include two rollers 400, 402 for engagement with the proximal end 302 of the elongate member 26. In some embodiments, the rollers 400, 402 may be moved apart from each other to allow loading of the elongate member 26, and may be moved towards each other to clamp the elongate member 26 in place. The rollers 400, 402 may be coupled to a drive assembly configured to move one or both of the rollers 400, 402 during use. In some embodiments, the drive assembly may be communicatively coupled to a controller, which receives an input signal from the workstation 2 (wherein the input signal is generated in response to a user input received at the workstation 2). The controller then provides an electronic signal in response to the input signal to actuate the drive assembly to move one or both of the rollers 400, 402. As shown in FIG. 17A, in some embodiments, the drive assembly may rotate one or both of the rollers 400, 402 in the direction 404 shown to thereby cause a translation of the elongate member 26 in the direction 406. In other embodiments, the drive assembly may reverse the rotation of the roller(s) to cause the elongate member 26 to move in a direction that is opposite from the direction 406. In some embodiments, when only one of the rollers 400, 402 is actuated, the other roller is passive (idle). Also, in some embodiments, one or more sensors may be coupled to the passive roller to detect slip between the elongate member and the passive roller. In some embodiments, when a slippage is detected by the slip-sensor(s), the system may stop moving the elongate member for safety purpose, and/or may warn a user of the slippage (e.g., by displaying a graphic on a screen, and/or emitting an audio signal).

Also, in some embodiments, the drive assembly may translate one or both of the rollers 400, 402 in the direction 410 shown in FIG. 17B to thereby cause a rotation of the elongate member 26 in the direction 412 shown. If only one of the rollers 400, 402 is translated, the other one of the rollers 400, 402 may be stationary (passive or idle). Alternatively, both rollers 400, 402 may be moved in opposite directions. In either case, the rollers 400, 402 may be considered as “moveable” relative to each other in opposite linear directions. In other embodiments, the drive assembly may reverse the direction of translation of the roller(s) to thereby cause the elongate member 26 to rotate in a direction that is opposite from the direction 412. Also, in some embodiments in which one of the rollers is passive, one or more sensors may be coupled to the passive roller to detect slip between the elongate member and the passive roller.

In some embodiments, the drive assembly is configured to provide rotational actuation and linear actuation for the rollers 400, 402 separately, and wherein the rollers 400, 402 are configured to maintain engagement with the elongate member 26 between the rotational actuation and linear actuation of the rollers 400, 402.

It should be noted that the number of rollers is not limited to two in the embodiments shown, and that the elongate member manipulator 24 may include more than two rollers in other embodiments. For example, as shown in FIG. 17C, in other embodiments, the manipulator 24 may include four rollers 400a, 400b, 402a, 402b. The rollers (e.g., either rollers 400a, 400b, rollers 402a, 402b, or all four rollers) may be rotated to cause the elongate member 26 to translate distally or proximally, and/or may be translated to cause the elongate member 26 to rotate in a clockwise or counter-clockwise direction, as similarly discussed with reference to FIGS. 17A and 17B.

Also, as shown in FIG. 17D, in other embodiments, the elongate member manipulator 24 may include two flexible members (drive belts) 420, 422 for engagement with the elongate member 26. The rollers (e.g., either rollers 400a-400d, rollers 402a-402d, or all of the rollers) may be rotated to turn the belts 420, 422 to cause the elongate member 26 to translate distally or proximally, and/or may be translated to translate the belt to cause the elongate member 26 to rotate in a clockwise or counter-clockwise direction, as similarly discussed with reference to FIGS. 17A and 17B. In some embodiments, the flexible member 420 and its corresponding rollers 400a-400d may be considered a “rotary member”. In such cases, the rotary member may have a rotational axis that is parallel to a rotational axis of any of the rollers 400a-400d, or the rotary member may be considered to have a rotational axis that is defined by any one of the rollers 400a-400d. Also, some embodiments, the flexible member 422 and its corresponding rollers 402a-402d may be considered a “rotary member”. In such cases, the rotary member may have a rotational axis that is parallel to a rotational axis of any of the rollers 402a-402d, or the rotary member may be considered to have a rotational axis that is defined by any one of the rollers 402a-402d.

Some embodiments of the elongate member manipulator 24 which may provide motorized actuation of the elongate member 26 (and/or other elongate instrument, such as a guidewire) in the manner described previously are described below. However, it should be noted that the manipulator 24 is not limited to the configuration described herein, and that the manipulator 24 may have other configurations in other embodiments. Many of the manipulator assemblies disclosed herein may be used to provide any motorized roll and insert or retraction actuation of any elongate instrument or member including but not limited to ablation probes, needles, scissors, clamps, forceps, graspers, guide wires, catheters, endoscopes, and other minimally invasive tools or surgical instruments.

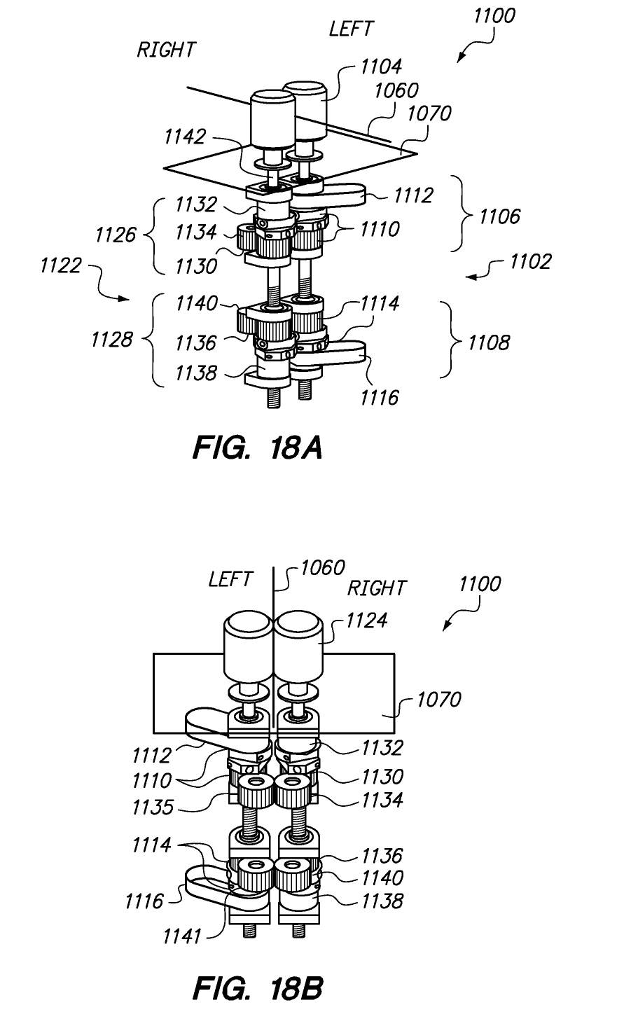

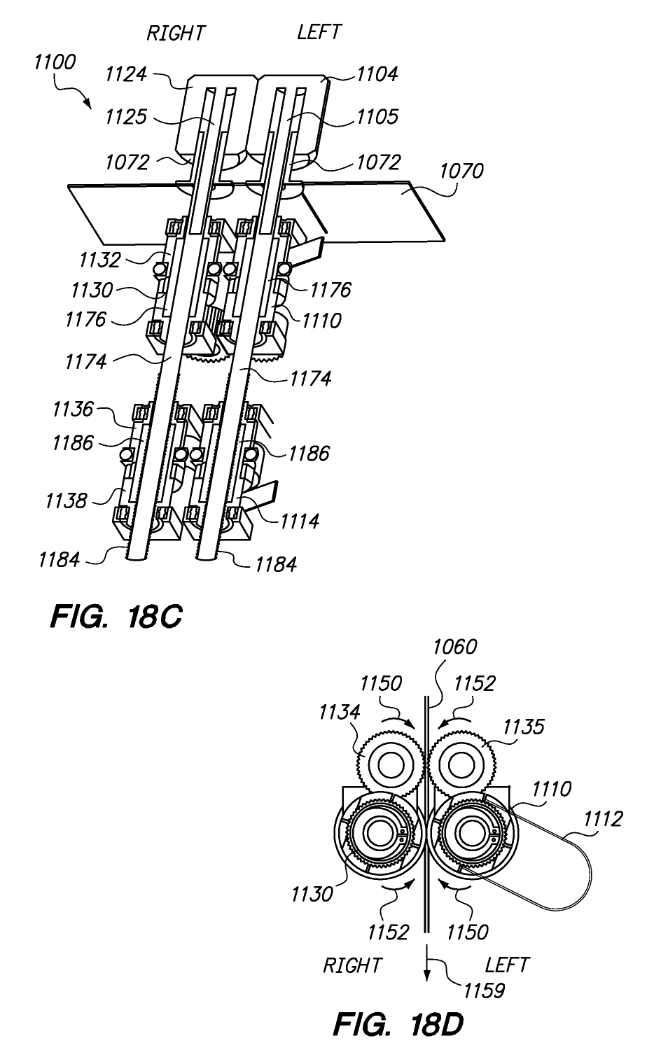

FIGS. 18A-18D illustrate different views of an elongate member manipulator 1100 in accordance with some embodiments. The elongate member manipulator 1100 may be an example of the elongate member manipulator 24. The elongate member manipulator 1100 includes a set of right and left motor actuated rotary members 1124, 1104. The rotary members can be used to robotically control the insertion and retraction of an elongate member 1060 (e.g., the elongate member 26, a guide wire, etc.) along a longitudinal axis of the elongate member 1060 and/or the roll or twist of the elongate member 1060 about a longitudinal axis of the elongate member 1060. In this variation, the rotary members are in the form of cylinders or feed rollers. However, the rotary members may include any other device suitable for providing rotary motion including belts.

As shown in FIG. 18A, the elongate member manipulator 1100 includes a right roller assembly 1122 and a left roller assembly 1102. Each roller assembly provides rotation and up-down or axial translation to their respective feed rollers 1124, 1104. The left roller assembly 1102 includes the left spline actuator 1106 and the left leadscrew actuator 1108. The right roller assembly 1122 includes a right spline actuator 1126 and a right leadscrew actuator 1128.

As illustrated in FIG. 18C, (a cross sectional view of the elongate member manipulator 1100), the internal elements of the left spline actuator 1106 may be identical to the internal elements of the right spline actuator 1126. Also, the internal components of the left leadscrew actuator 108 may be identical to the internal components of the right leadscrew actuators 1128. Thus both right and left spline actuators 1126, 1106 may include a spline shaft 1174, coupled to a spline nut 1176 which is driven by a gear train which will be described in further detail below. Similarly, the right and left leadscrew actuators 1128, 1108 may include a leadscrew shaft 1184, coupled to a leadscrew nut 1186, driven by a similar gear train.

The spline nut 1176 and leadscrew nut 1186 may be sized such that two axially adjacent gears can create a gear stack that covers the entire axial length of each nut. Thus the left spline actuator 1106 may include a left spline gear stack 1110, which acts as one gear driving the spline shaft 1174 which in turn drives the left roller 1104. The left leadscrew actuator 1108 may also have a similar left leadscrew gear stack 1114 which functions in a similar manner. In alternative variations, a smaller spline nut and smaller leadscrew nut may be utilized allowing for a single gear to be used as opposed to a gear stack.

The right roller assembly 122 may include gears that are driven (in a manner as will be described below), and instead of stacking two adjacent gears, the right spline actuator 1126 can include a smooth shaft 1132 and a right spline output gear 1130. The right leadscrew actuator 1128 can include a smooth shaft 1138 and a right leadscrew output gear 1136. The right spline output gear 1130 and right leadscrew output gear 1136 are coupled to the spline shaft 1174 and leadscrew shaft 1184 respectively and the gears drive the motion of the roller 1124.

In operation, the right and left rollers 1124, 1104 may rotate at substantially the same rate but in opposite directions to facilitate insertion or retraction of an elongate member 1060 (shown in FIGS. 18A-18B and 18D). Idler gears may be used to couple the motion of the right and left actuator assemblies 1122, 1102.

As shown in FIG. 18B the elongate member manipulator 1100 may include a right spline coupling gear 1134, a left spline coupling gear 1135, a right leadscrew coupling gear 1140 and a left leadscrew coupling gear 1141. To rotate the rollers 1104, 1124, the left spline gear stack 1110 is driven by a spline belt 1112, which in turn can be directly driven by a motor or driven indirectly by a series of gears, belts or pulleys (not shown). As previously described, this rotation will cause a direct rotation of the left roller 1104. Simultaneously, the left spline gear stack 1110 may use the coupling gears to drive the right roller 1124 in an opposite direction to that of the left roller 1104.

FIG. 18D, shows a top view of the elongate member manipulator 1100 (the feed rollers are not shown for clarity). In this example, the left spline gear stack 1110 is driven in the CW direction 1150, the left spline coupling gear 1135 will rotate in the CCW direction 1152, rotating the right spline coupling gear 1134 in the CW direction 1150, and the right spline output gear 1130 in the CCW direction 1152. If all the gears are sized equally, the left spline gear stack 1110 and right spline output gear 1130 will rotate at the same rate in opposite directions, rotating the rollers 1104, 1124 at equal rates in opposite directions, which would drive the guide wire 1060 in a forward propelling motion 1159. Reversing the direction of the spline belt 1112 would reverse the directions of both the left spline gear stack 1110 and right spline output gear 1130, and as a result, reverse the direction of rotation of the rollers 104, 124, thereby driving the elongate member 1060 in the reverse propelling motion.

The leadscrew actuators 1108, 1128 may function in a similar manner but alternatively cause one roller to translate upwards while the other roller translates downwards at a substantially similar rate. This motion will drive the elongate member 1060 in a roll or torque motion. The clockwise or counterclockwise directions of roll are dependent on the direction of rotation of the leadscrew belt 1116. Both insert/propelling motion and roll/torque motion can be accomplished with varying speed rates for each axis. The propelling and torque axes motions can be simultaneous, or they can be independent of each other.

FIG. 20 illustrates a cross sectional view of one variation of a roller actuator 1170 that may be utilized to provide motorized rotation and translation actuation of one or more rotary members, such as a feed roller. Such a roller actuator may be utilized to provide rotation and translation actuation of various rollers, including, for example, the rollers of elongate member manipulator 1100 described above.

The roller actuator 1170 includes a one or more spline actuators 1172 having a spline shaft 1174 coupled to a spline nut 1176 mounted on spline nut bearings 1178. The spline nut 176 is rotated by a spline gear 1180 which can either be directly motor driven or indirectly motor driven via a series of gears, belts or pulleys (not shown). The spline shaft 1174 may be fixably coupled to a rotary member such as a feed roller 1104, so that the rotation of the spline nut creates rotation of the feed roller. A single leadscrew actuator 1182 which includes a leadscrew shaft 1184, leadscrew nut 1186, leadscrew nut bearings 1188, and a leadscrew gear 1190 is provided adjacently below the spline actuator 1172 to provide up-down translation of a feed roller. The leadscrew nut 1186 is driven by the leadscrew gear 1190 which can either be directly motor driven or indirectly motor driven via a series of gears, belts or pulleys (not shown). Rotation of the leadscrew nut 1186 lifts and lowers the leadscrew shaft 1184 and spline shaft 1174, creating the up and down lift or axial translation of the feed roller.

In certain variations, the spline shaft 1174 and leadscrew shaft 1184 may be coupled so that rotation of one may cause rotation of the other. Because the spline shaft 1174 is constructed as a spline, it can be driven up and down by the leadscrew shaft 1184 without lifting the spline nut 1176, spline bearings 1178, or spline gear 1180. To actuate only rotation of the feed roller, both spline nut 1176 and leadscrew nut 1186 may be rotated at the same rate. As a result, the leadscrew shaft 1184 will rotate at the same rate as the leadscrew nut 1186 so that no lift motion will occur. To actuate only lift of a feed roller, the leadscrew nut 1186 may be rotated without movement of the spline nut 1176. Alternatively, simultaneous rotational and translational motion of a feed roller may be provided by slowing and speeding up the leadscrew nut 1186 relative to the spline nut 1176 or vice versa.

In an alternative variation, the spline shaft 1174 and the leadscrew shaft 1184 may not be coupled so that movement of the spline actuator 1172 and the leadscrew actuator 1182 are completely independent. Alternatively, the spline shaft 1174 and leadscrew shaft 1184 could be free to rotate independently by joining the two shafts in a ball and socket type configuration. Additional bearing support may be utilized in such a variation.

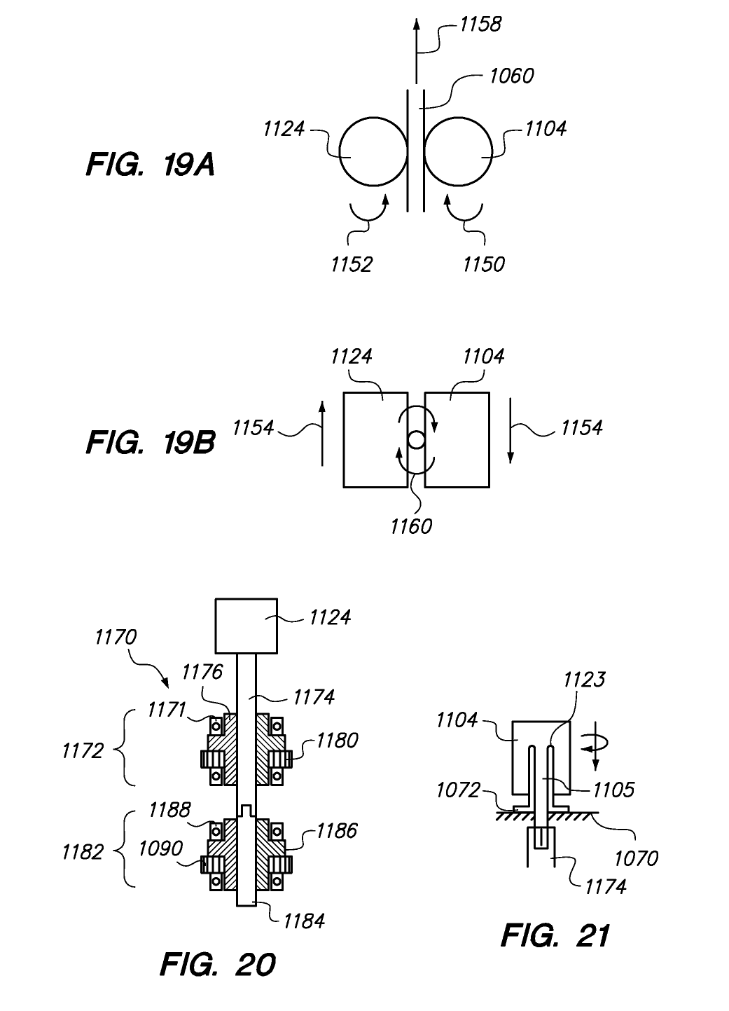

FIGS. 19A-19B illustrate examples of feed rollers in use, showing how an elongate member 1060 may be actuated by the feed rollers 1124, 1104. FIG. 19A illustrates a top view of a pair of feed rollers 1124, 1104 illustrating how the feed rollers can rotate about their axes in opposite directions 1152, 1150 to drive the elongate member 1060 in a backwards propelling motion or a retract motion 1158. The feed rollers can also be rotated in opposing directions to provide forward propelling or insert motion (not shown). FIG. 19B shows a front view of the feed rollers 1124, 1104 illustrating how the feed rollers can translate axially along their axes in opposite translation directions 1154 to torque or roll 1160 the elongate member 1060.

Forward or reverse insert/retract motion 1158 is dependent on the direction of rotation 1152, 1150 of the rollers 1124, 1104 while clockwise or counter-clockwise roll motion 1160 is dependent on the direction of up and down linear or axial translation 1154 of the rollers 1124, 1104. Both insert/retract motion and roll motion can be accomplished with varying speed rates for each axis. The insert and roll actuations can be independent of one another, or they may occur simultaneously. Also simultaneous roll and insert actuation can be desirable in part because traditional manual procedures are performed in that manner. Currently physicians articulate and steer manual guidewires by inserting and rolling simultaneously resulting in more of a spiraling insertion. It can be desirable for robotic systems to emulate manual procedures for physician ease of use.

In alternative variations, insert motion can be provided by feed rollers while roll motion actuation may be provided by clamping the elongate member 1060 in a clamp mechanism and rolling the clamp mechanism. In this variation roll and insert motion may be alternated between insert and roll with typical clutching mechanisms that release grip from one actuator assembly while the alternate assembly provides actuation. For example, in a feed roller variation with clutching, feed rollers used to actuate insert may release the elongate member 1060 while actuators providing rotation to roll the elongate member 1060. The release of the elongate member 1060 from one actuator during activation of the alternate actuator in systems which use feed rollers for insert but roll the elongate member 1060 with a separate mechanism allows the elongate member 1060 to overcome friction experienced from the feed rollers during roll actuation. If insert and roll are simultaneously actuated the elongate member 1060 may be gripped in the insert feed rollers which could result in the stripping or winding up the elongate member 1060.

Systems which clutch between insert and roll actuators typically release grip of the elongate member 1060 by one actuator to allow the alternate actuator to grip the elongate member 1060. By releasing the elongate member 1060, any tracking of elongate member 1060 position using encoders may be lost which could decrease the accuracy of position tracking. Also, additional actuators may result in a more complex or more costly system.

In certain variations, the elongate member 1060 may be loaded into the elongate member manipulator 1100 by being back or front loaded or fed into the feed rollers 1104, 1124 while rotating the feed rollers 1104, 1124 in an insert or retract motion.

In certain variations, the elongate member manipulator 1100 may be designed such that at least a portion of the elongate member manipulator 1100 remains in a sterile field. For example, the motors and drive mechanisms or drive components of the elongate member manipulator 1100 may be situated in a non-sterile field and a sterile drape could be placed in-between the drive components and the feed rollers. Thus, the elongate member 1060 held by the feed rollers will remain sterile for insertion into a patient's anatomy. In certain variations, components of an elongate member manipulator 1100 which are meant to remain sterile may be disposable and/or the complexity of such components may be minimized in order to minimize or reduce overall costs of such disposable components or the elongate member manipulator 1100.

Referring back to FIGS. 18A-18C, one variation of a sterile drape 1070 used to create a sterile field that includes the feed rollers 1104, 1124 and the elongate member 1060 is illustrated. All other components could be positioned in a non-sterile field.

FIG. 21 shows an example of the sterile drape 1070 installed between the left feed roller 1104 and the spline shaft 1174. The sterile drape 1070 may be designed such that the roller 1104 can be removeably replaceable where the drape 1070 could be placed over the spline shaft 1174 and the rollers could be installed over the drape in the sterile field. The sterile drape 1070 could have a sterile drape bushing 1072 that is fixably attached to the drape 1070. The roller 1104 could be coupled to the bushing 1072 via a roller shaft 1105 extending through the bushing 1072 which is coupled to the spline shaft 1074 in the non-sterile field. The roller shaft 1105 and spline shaft 1074 could be coupled by keying each shaft to mate, thus allowing rotation of the spline shaft 1074 to cause a one to one rotation of the roller shaft 1105. The key can be shaped as a hexagon, triangle, star, cross or any other shape. The roller 1104 may rotate relative to the bushing and may translate up and down like a piston. A fastener may be provided to secure the roller 1104 in place to prevent slippage in the axial direction. Alternatively, the roller shaft 1105 may be threaded and coupled to a threaded hole in the spline shaft 1174. As the roller 1104 moves up and down, a left roller groove 1123 on the roller may create a labyrinth seal and maintain a sterile boundary between the bushing 1072 and roller 1104. Optionally, an o-ring or lip seal can be placed between the bushing 1072 and roller 1104 to prevent fluid ingress and create an improved sterile boundary. The sterile drape 1070 could provide for a sterile interface for the right feed roller 1124 in the same manner.

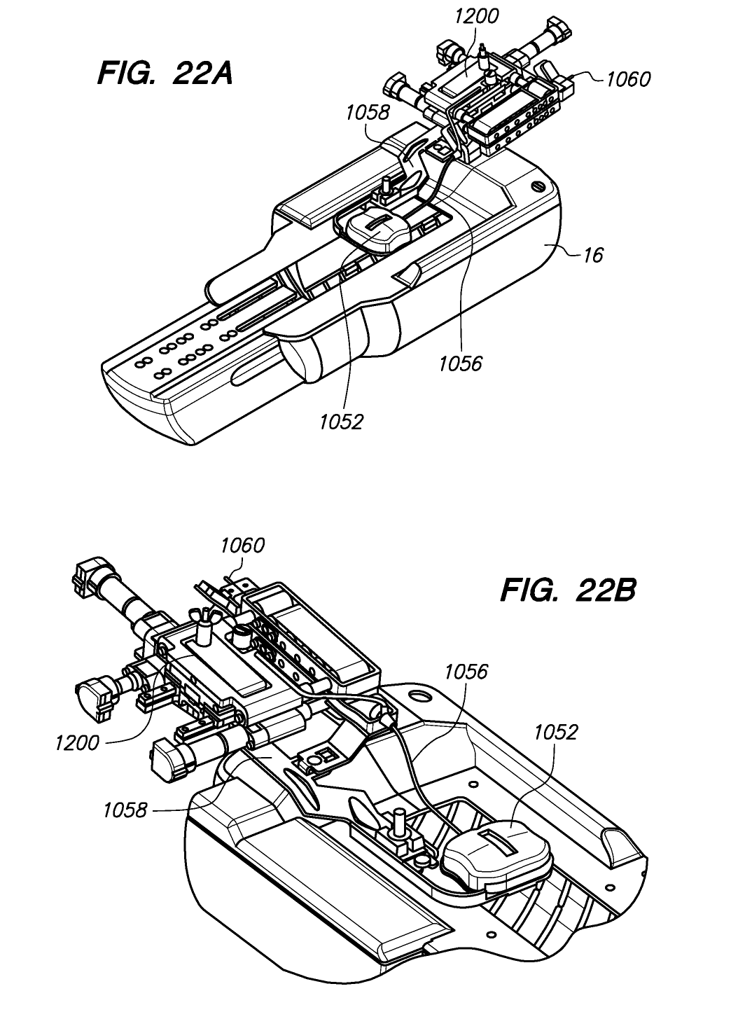

FIGS. 22A-22B illustrate another variation of an elongate member manipulator 1200 which includes rotary members in the form of belts. The elongate member manipulator 1200 may be an example of the elongate member manipulator 24. The elongate member manipulator 1200 is shown mounted on an instrument driver 16. The elongate member manipulator 200 may by utilized to feed an elongate member 1060 co-axially into a guide catheter splayer 1052. The elongate member 1060 may be fed into a support tube 1056 which subsequently feeds into the guide catheter splayer 1052, and ultimately into a guide catheter (not shown). In certain variations, the elongate member manipulator 1200 may be mounted on the instrument driver along with a guide and/or a sheath splayer/catheter or the elongate member manipulator 1200 may be mounted alone. Optionally, the elongate member manipulator 1200 may be utilized to feed the elongate member 1060 co-axially into a sheath and or catheter. Optionally, the elongate member manipulator 1200 may be utilized to feed the elongate member 1060 directly into a patient's body or anatomy.

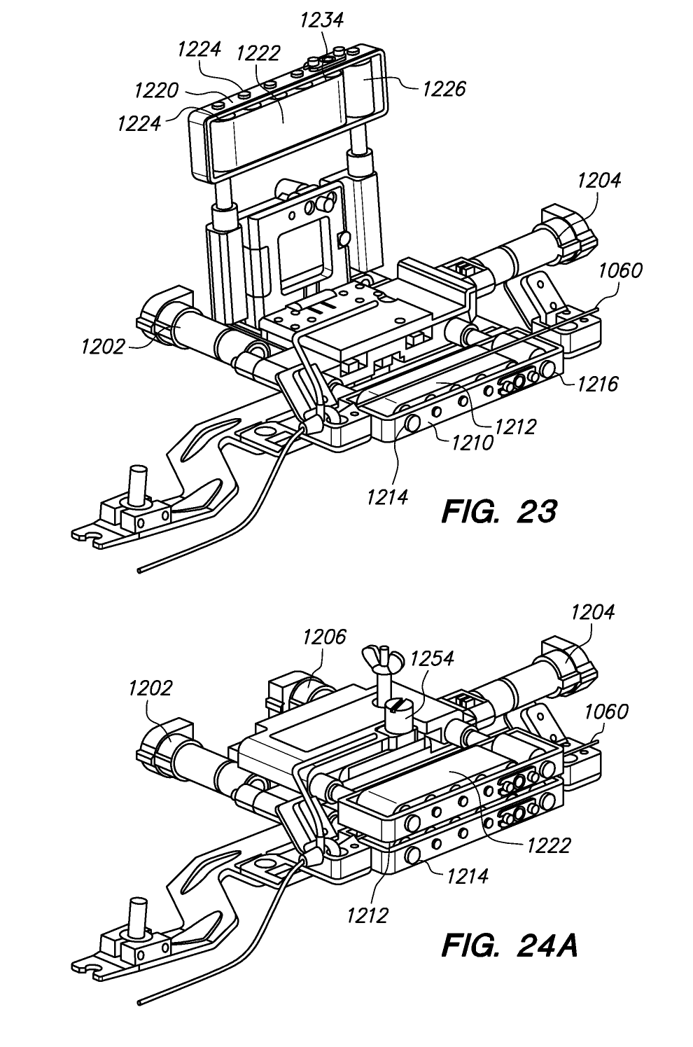

FIG. 23 illustrates the elongate member manipulator 1200 in an open hinged configuration. The elongate member manipulator 1200 may include a drive assembly and an elongate member holder. The components of the elongate member holder include a drive belt assembly 1210 and an idler belt assembly 1220. Both belt assemblies include belts 1212, 1222 with pulleys 1214, 1224. The drive pulley 1084 may be directly driven by an insert servo motor 1102 or other mechanism to turn the drive belt 1212. The idler (passive) belt 1222 is free to rotate about the idler pulley 1224. The belts may be constructed from various materials known to person having ordinary skill in the art. The belts may have various dimensions. For example, about 1″ wide Texin® or silicon rubber, durometer 90A profiled timing belts may be utilized covering a length of about 4.5″ from opposite outer diameter edges of the belt. Other variations may use alternative widths, other dimensions, and materials with alternative durometers for the belts. In one variation the belts can be constructed from any gamma sterilizable material which is well known in the art including but not limited to thermoplastics such as ABS or PET, fluoropolymers such as polyvinyl fluoride, polymides, polystyrenes, polyurethanes, polyesters, or polyesters. Optionally, bands or feed rollers could be used in place of belts.

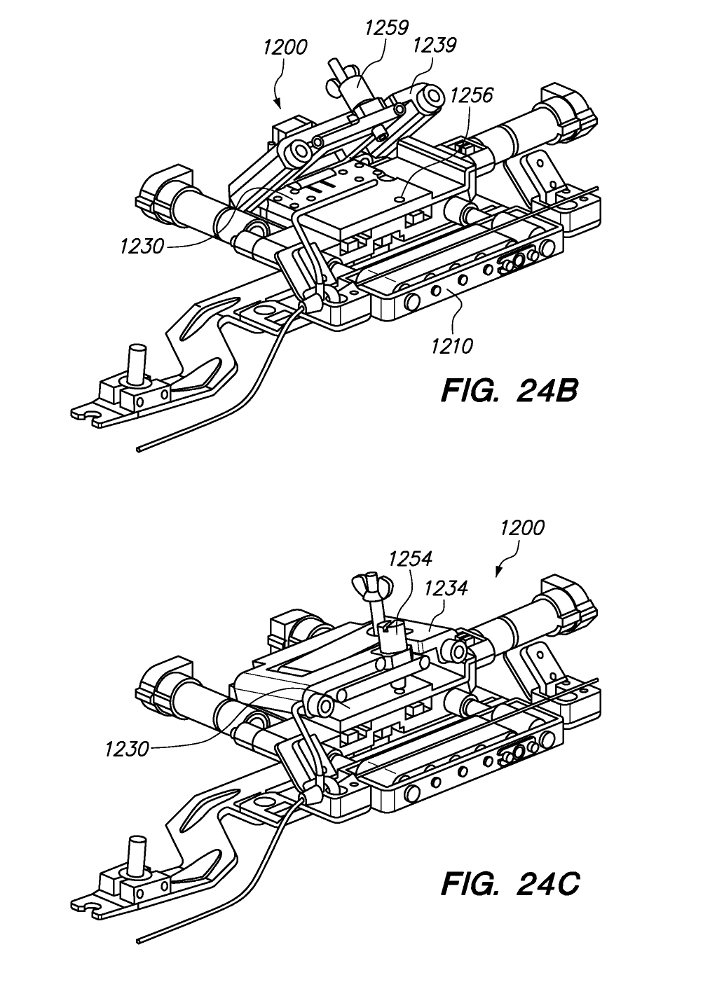

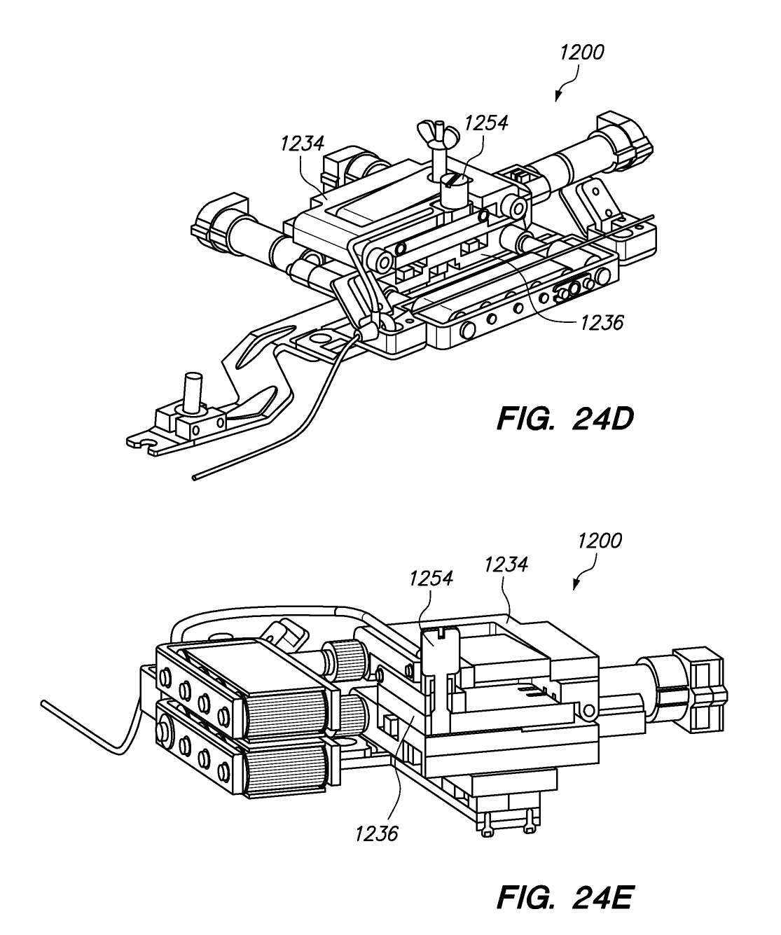

As shown in FIGS. 24A-24C, the drive assembly can include an upper slide assembly 1233, a lower slide assembly 1230, an insert motor 1202, and a roll motor 1204, as well as a set of rails, a rack and a pinion (not shown here but described in detail below). In use, as illustrated in FIGS. 23 and 24A, the upper slide assembly 1234 can hinge open a plurality of degrees for workflow clearance, the elongate member 1060 can be placed on the drive belt 1212, and the elongate member manipulator 1200 or system can be closed so that the elongate member 1060 is held between the drive belt 1212 and the idler belt 1222. This allows the elongate member 1060 to be loaded into the elongate member manipulator 1200 anywhere along the length of the elongate member 1060, which may expedite the loading procedure instead of being restricted to load the elongate member 1060 by feeding the elongate member 1060 from the back of the system. Also the elongate member 1060 may be loaded when the belts are in any position. For example, the drive belt 1212 may be at an arbitrary position such that the drive motor 1202 does not require any type of initialization or homing before installation of the elongate member 1060. Additionally the elongate member 1060 may be removed from the elongate member manipulator 1200 or system mid procedure if the operator desires to switch from using the robotic manipulator to manual control of the elongate member 1060.

In other embodiments, the elongate member 1060 may be backloaded into the manipulator 1200. A back loaded elongate member 1060 would be retracted or pulled out of a patient's body before removing the elongate member 1060 from the manipulator 1200 to switch to manual control.

To ensure that the upper slide assembly 1234 and lower slide assembly 1230 stay closed during operation, a captive screw 1254 can be used. A variation including a captive screw 1254 is shown in FIGS. 24B-24E which illustrate an isometric view of the elongate member manipulator 1200 with only the drive belt assembly 1210 shown (idler belt assembly not shown for clarity). FIG. 24B illustrates the elongate member manipulator 1200 in an open position, FIG. 24C illustrates the elongate member manipulator 1200 as it is partially closed, and FIG. 24D shows the elongate member manipulator 1200 closed and locked. The captive screw 1254 remains captive with the upper slide assembly 1234 and locks into a threaded hole 1256 in the lower slide assembly 1230. FIG. 24E illustrates a cross section of the elongate member manipulator 1200 illustrating the operation of the captive screw 1254. In alternative variations, a latch, fastener or other type of locking, fastening or latching mechanism may be used instead of a captive screw.

As illustrated in FIG. 24A, once the elongate member 1060 is loaded and held between the drive belt 1212 and the idler belt 1222, the insert motor 1202 drives the drive pulley 1214, turning the drive belt 1212 and propelling the elongate member 1060 forward or backwards (insert or retract) depending on the rotational direction of the motor and pulley. With sufficient frictional pinching, gripping, pressing, or holding force holding the guide wire 1060 between the drive belt 1212 and idler belt 1222, the idler belt 1222 will turn at the same rate as the drive belt 1212, and the belts will hold the guide wire 1060 such that lateral linear movement or displacement of the elongate member 1060 relative to the belts may be eliminated, minimized or reduced.

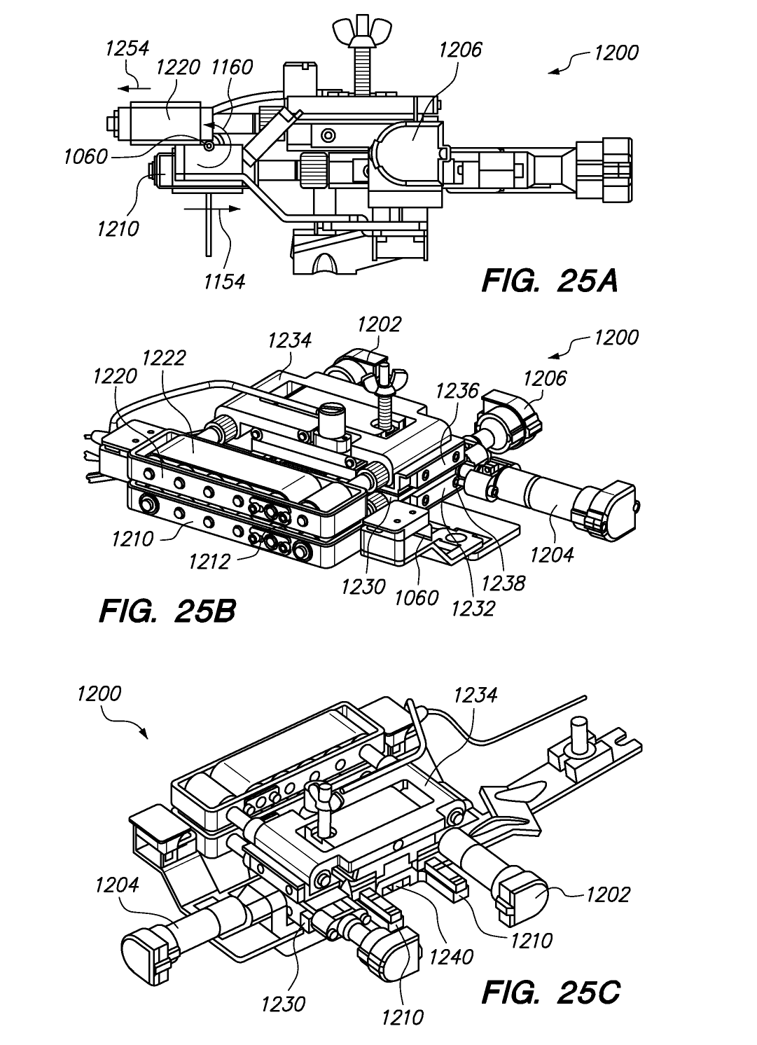

FIGS. 25A-25C illustrate various views of the elongate member manipulator 1200 showing various components of the elongate member manipulator that function to provide roll actuation of the elongate member 1060. (Some components of the elongate member manipulator 1200 are hidden for clarity.)

FIG. 25A illustrates an end view of the elongate member manipulator 1200. FIGS. 25B-25C illustrate perspective views of the elongate member manipulator 1200 providing different angles showing the lower slide assembly 1230 and the upper slide assembly 1234. The lower slide assembly 1230 and upper slide assembly 1234 may each be attached to linear rails 1240. The lower slide assembly 1230 includes the insert motor 1202 and a slip detection encoder 1204. The drive belt assembly 1210 attaches to the lower slide assembly 1230 while the idler belt assembly 1220 attaches to the upper slide assembly 1234. Both lower and upper assemblies 1230, 1234 have a rack 1232, 1236 that is coupled to a pinion 1238 driven by a roll motor 1206. The roll motor 1204 is mounted stationary relative to the instrument driver so that when the pinion 1238 is turned, the slide assemblies 1230, 1234 move or translate in opposing directions, driving both the drive belt assembly 1210 and the idler belt assembly 1220 in opposing translational directions 1154. This motion will roll, rotate or torque the elongate member 1060 as shown by the arrow 1160. Translation of the drive belt assembly 1210 and idler belt assembly 1220 in directions opposite those shown in FIG. 25A would result in roll of the elongate member 1060 in the direction opposite that of arrow 1160.

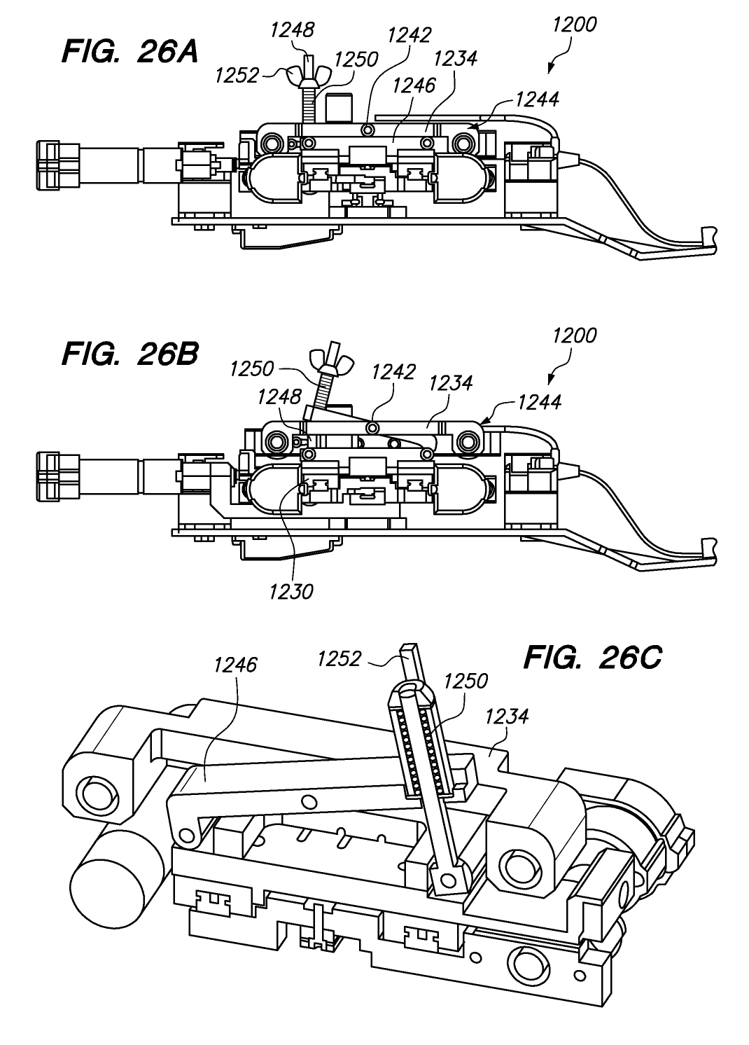

Thus, in certain variations, the upper slide assembly 1234 of the elongate member manipulator 1200 may include a hinge 1242 and a suspension mechanism 1244. FIGS. 26A-26B show a left side view of an elongate member manipulator 1200, with the suspension mechanism 1244 in an open and closed configuration respectively, while FIG. 26C shows a cross section of the assembly 1234 with the suspension mechanism 1244. The suspension mechanism 1244 may include a lever arm 1246, a lever shaft 1248, a lever spring 1250 and a tightening nut 1252. The suspension mechanism 1244 may provide a mechanism by which the force applied by the lever spring 1250 to hold the guide wire between the idler belt assembly 1220 and the drive belt assembly 1210 may be adjusted in order to accommodate a variety of elongate member diameters while providing sufficient pinching force for a variety of elongate member diameters.

As illustrated in FIG. 26B, the tightening nut 1252 may be used to control the swing of the lever arm 1246 to adjust the grip force between the upper slide assembly 1234 and the lower slide assembly 1230 to apply the necessary grip force for various elongate member diameters and to provide an increased force ratio for elongate member compression. By way of example but not limitation, if a 2 to 1 force ratio could be applied where a 20 lb elongate member load was required, a 10 lb spring would be applied to the lever. The range of elongate member diameters that could be accommodated for this example may range from about 0.014″-0.038″. In other embodiments, the elongate member 1060 may have a cross sectional size that is larger than that described.

In some embodiments, both the sheath catheter assembly 62 and guide catheter assembly 61 may be mounted on separate carriages that are motor actuated to provide a propelling motion in the insert and retract directions of the guide catheter 61a and sheath catheter 62a. In one variation, the elongate member manipulator 24 is fixably mounted to the same carriage as the guide catheter assembly 61. By mounting the elongate member manipulator 24 in this fashion, buckling of the elongate member 26 may be minimized by locating the elongate member manipulator 24 as close to the proximal end of the guide catheter 61a as possible and/or maintaining a constant gap between the elongate member manipulator 24 and guide catheter 61a proximal end. The constant gap also avoids an inadvertent collision between the elongate member manipulator 24 and the guide catheter assembly 61. In other embodiments, the elongate member manipulator 24 may be mounted to other areas at the robotic instrument driver 16.

The elongate member manipulator 24 is not limited to having the configuration/features described herein, and may have other configurations/features in other embodiments. Elongate member manipulators that may be used with the robotic system 10 have been described in U.S. patent application Ser. No. 13/173,994, which was previously incorporated by reference.

Although the elongate member manipulator 24 (e.g., manipulator 1100, 1200) has been described with reference to moving the elongate member 26 (which may be an energy delivery device, or a guidewire), in other embodiments, the manipulator 24 may also be used to move multiple elongate members. For example, in other embodiments, during a procedure, the manipulator 24 may be employed to move a guidewire (an elongate member 26) for placement of the catheter 61a and/or the sheath 62a. After the distal end of the catheter 61a and/or the distal end of the sheath 62a is desirably positioned inside the patient, the guidewire may be removed from the manipulator 24, and a treatment device (another elongate member 26) may then be inserted into the lumen of the catheter 61a, and the proximal end 302 of the treatment device may then be removably mounted to the manipulator 24. The manipulator 24 then positions the treatment device inside the patient until its distal end 300 is placed at a desired target location. The treatment device may then be used to perform a procedure, such as a treatment procedure to treat tissue.

IV. Driving Modes

As discussed, the system 10 may be configured to move the sheath 62a distally or proximally, move the catheter 61a distally or proximally, and to move the elongate member 26 distally or proximally. In some cases, the movement of the sheath 62a may be relative to the catheter 61a, while the catheter 61a remains stationary. In other cases, the movement of the catheter 61a may be relative to the sheath 62a while the sheath 62a remains stationary. Also, in other cases, the sheath 62a and the catheter 61a may be moved together as a unit. The elongate member 26 may be moved relative to the sheath 62a and/or the catheter 61a. Alternatively, the elongate member 26 may be moved together with the sheath 62a and/or the catheter 61a.

In some embodiments, the workstation 2 is configured to provide some or all of the following commanded motions (driving modes) for allowing the physician to choose. In some embodiments, each of the driving modes may have a corresponding button at the workstation 2 and/or the bedside control 402.

Elongate Member Insert

When this button/command is selected, the manipulator 24 inserts the elongate member 26 at a constant velocity.

Elongate Member Roll

When this button/command is selected, the manipulator 24 rolls the elongate member 26 at a constant angular velocity

Elongate Member Size

When the size or gauge of the elongate member 26 is inputted into through the user interface, the system will automatically alter roll and insert actuation at the proximal end of the elongate member 26 accordingly to achieve desired commanded results. In one implementation, when a user inputs the elongate member's size, the system automatically changes its kinematic model for driving that elongate member 26. So if the user commands the elongate member 26 to move to a certain position, the system will calculate, based on the kinematic model, roll and insert commands, which may be different for different elongate member sizes (e.g., elongate members 26 with different diameters). By inputting the elongate member's size, the system knows which kinematic model to use to perform the calculation. Such feature is beneficial because different sized elongate members 26 behave differently.

Leader/Sheath Select

When this button/command is selected, it allows the user to select which device (e.g., catheter 61a, sheath 62a, elongate member 26, or any combination of the foregoing) is active.

Leader/Sheath Insert/Retract

When this button/command is selected, the instrument driver assembly inserts or retracts the catheter 61a/sheath 62a while holding the elongate member 26 and any non-active device fixed relative to the patient. When this motion causes the protruding section of the catheter 61a to approach zero (due to insertion of the sheath 62a or retraction of the catheter 61a), the system automatically relaxes the catheter 61a as part of the motion.

Leader/Sheath Bend

When this button/command is selected, the instrument driver assembly bends the articulating portion of the catheter 61a/sheath 62a within its currently commanded articulation plane.

Leader/Sheath Roll

When this button/command is selected, the instrument driver assembly uses the pullwires to “sweep” the articulation plane of the device (catheter 61a and/or sheath 62a) around in a circle through bending action of the device. Thus, this mode of operation does not result in a true “roll” of the device in that the shaft of the device does not roll. In other embodiments, the shaft of the device may be configured to rotate to result in a true roll. Thus, as used in this specification, the term “roll” may refer to an artificial roll created by seeping a bent section, or may refer to a true roll created by rotating the device.

Leader/Sheath Relax

When this button/command is selected, the instrument driver assembly gradually releases tension off of the pullwires on the catheter 61a/sheath 62a. If in free space, this results in the device returning to a straight configuration. If constrained in an anatomy, this results in relaxing the device such that it can most easily conform to the anatomy.

Elongate Member Lock

When this button/command is selected, the elongate member 26 position is locked to the catheter 61a position. As the leader is articulated or inserted, the elongate member 26 moves with the catheter 61a as one unit.

System Advance/Retract

When this button/command is selected, the instrument driver assembly advances/retracts the catheter 61a and sheath 62a together as one unit. The elongate member 26 is controlled to remain fixed relative to the patient.

Autoretract

When this button/command is selected, the instrument driver assembly starts by relaxing and retracting the catheter 61a into the sheath 62a, and then continues by relaxing and retracting the sheath 62a with the catheter 61a inside it. The elongate member 26 is controlled to remain fixed relative to the patient.

Initialize Catheter

When this button/command is selected, the system confirms that the catheter 61a and/or the sheath 62a has been properly installed on the instrument driver assembly, and initiates pretensioning. Pretensioning is a process used to find offsets for each pullwire to account for manufacturing tolerances and the initial shape of the shaft of the catheter 61a and/or the sheath 62a.

Leader/Sheath Re-Calibration

When this button/command is selected, the instrument driver assembly re-pretensions the catheter 61a and/or the sheath 62a in its current position. This gives the system the opportunity to find new pretension offsets for each pullwire and can improve catheter driving in situations where the proximal shaft of the catheter 61a has been placed into a significant bend. It is activated by holding a relax button down for several seconds which ensures that the device is fully de-articulated. Alternatively the re-calibration may be activated without holding down the relax button to de-articulate the device.

Leader Relax Remove

When this button/command is selected, the instrument driver assembly initiates a catheter removal sequence where the catheter 61a is fully retracted into the sheath 62a, all tension is released from the pullwires, and the splayer shafts (at the drivable assembly 61 and/or drivable assembly 62) are driven back to their original install positions so that the catheter 61a can be reinstalled at a later time.

Leader Yank Remove

When this button/command is selected, the instrument driver assembly initiates a catheter removal sequence where the catheter 61a is removed manually.

Emergency Stop

When this button/command is selected, the instrument driver assembly initiates a gradual (e.g., 3 second) relaxation of both the catheter 61a and the sheath 62a. The components (e.g., amplifier) for operating the catheter 61a, elongate member 26, or another device are placed into a “safe-idle” mode which guarantees that no power is available to the motors that drive these elements, thereby bringing them rapidly to a stop, and allowing them to be manually back-driven by the user. Upon release of the emergency stop button, the system ensures that the catheter 61a is still in its allowable workspace and then returns to a normal driving state.

Segment Control:

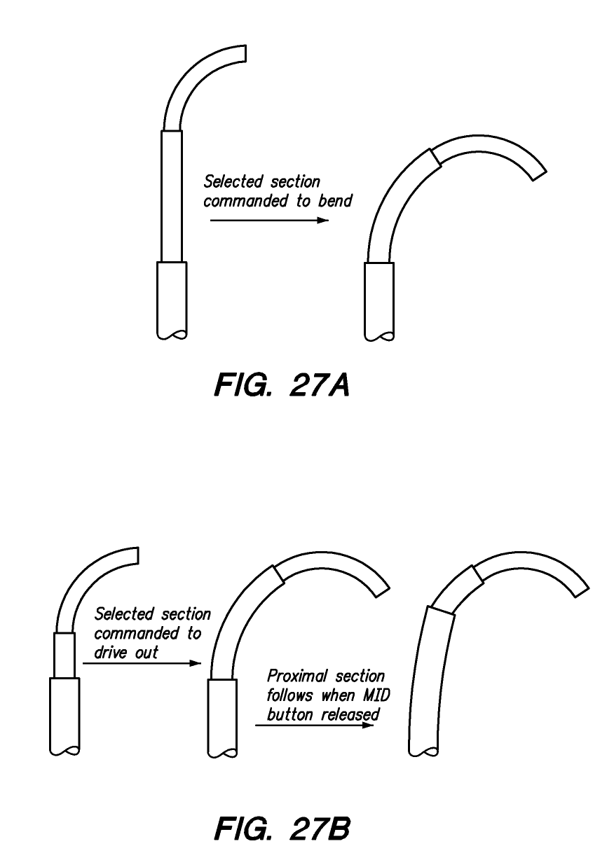

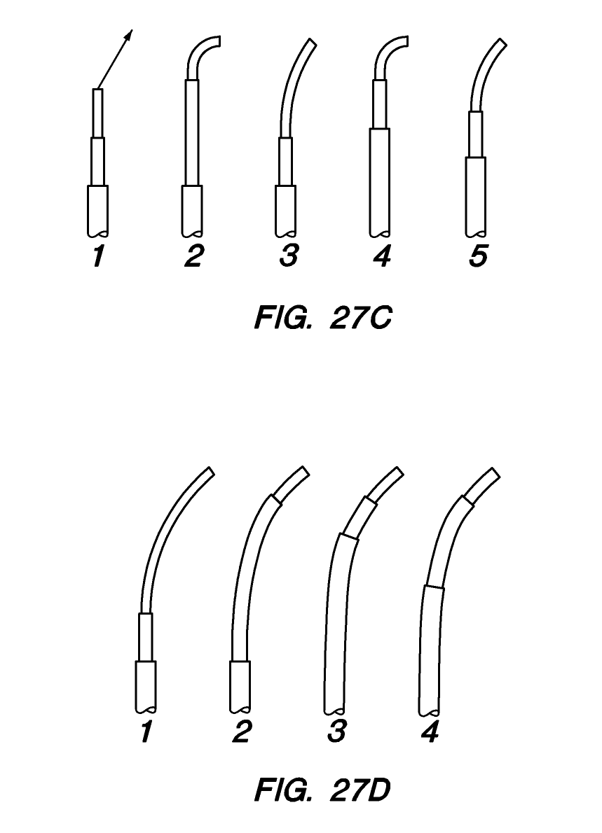

In some embodiments, the workstation 2 allows a user to select individual segment(s) of a multi-segment catheters (such as the combination of the catheter 61a and the sheath 62a), and control each one. The advantage of controlling the catheter in this way is that it allows for many options of how to control the movement of the catheter, which may result in the most desirable catheter performance. To execute this method of catheter steering, the user selects a segment of the catheter to control. Each segment may be telescoping or non-telescoping. The user may then control the selected segment by bending and inserting it using the workstation 2 to control the position of the end point of the catheter. Other segment(s) of the catheter will either maintain their previous position (if it is proximal of the selected section) or maintain its previous configuration with respect to the selected section (if it is distal of that section) (FIG. 27A).

Follow Mode:

In some embodiments, the workstation 2 allows the user to control any telescoping section while the more proximal section(s) follows behind automatically. This has the advantage of allowing the user to focus mostly on the movement of a section of interest while it remains supported proximally. To execute this method of catheter steering, the user first selects a telescoping section of the elongate instrument (e.g., catheter 61a and sheath 62a) to control. This section is then controlled using the workstation 2 to prescribe a location of the endpoint of the segment. Any segment(s) distal of the section of interest will maintain their previous configuration with respect to that section. When the button on the workstation 2 is released, any segment(s) proximal of the section of interest will follow the path of the selected section as closely as possible until a predefined amount of the selected section remains (FIG. 27B). As an alternative to this driving mode, the segment(s) of the elongate instrument which is proximal of the section of interest could follow along as that segment is moved instead of waiting for the button to be released. Furthermore, with either of these automatic follow options, the system may optionally be configured to re-pretension the sections that have been driven out and re-align the sections that are proximal of the driven section.

Follow mode may be desirable to use to bring the more proximal segments of the elongate instrument towards the tip to provide additional support to the distal segment. In cases where there are three or more controllable sections of the elongate instrument, there are several options for how to execute a “follow” command. Consider the example in FIG. 27D where the distal segment (which may be a guidewire or a steerable instrument in some embodiments) has been driven out as shown in frame 1. The “follow” command could be executed by articulating and/or inserting only the middle segment (which may be the catheter 61a in some embodiments) of the elongate instrument as shown in frame 2. The “follow” command could be executed by articulating and/or inserting only the most proximal segment (which may be the sheath 62a in some embodiments) of the elongate instrument as shown in frame 3. The “follow” command could also be executed by coordinating the articulation and/or insertion of multiple proximal segments of the elongate instrument as shown in frame 4. Combining the motion of multiple sections has several potential advantages. First, it increases the total degrees-of-freedom available to the algorithm that tries to fit the shape of the following section(s) to the existing shape of the segment being followed. Also, in comparison to following each segment sequentially, a multi-segment follow mode simplifies and/or speeds up the workflow. In addition, multi-segment increases the distance that can be followed compared to when only one proximal segment is used to follow the distal segment.

Mix-and-Match Mode:

In some embodiments, the workstation 2 allows the user to have the option of mixing and matching between articulating and inserting various sections of a catheter. For example, consider the illustration in FIG. 27C, and assuming that the distal most section of the elongate instrument is the “active” segment. If the user commands a motion of the tip of the elongate instrument as indicated by the arrow in Frame 1, there are several options available for how to achieve this command: (1) Articulate and extend the “active” segment, which is illustrated in frame 3 and is likely considered the normal or expected behavior; (2) Articulate the active distal most segment and insert one of the other proximal segments, as illustrated in frames 2 and 4; (3) Articulate the active distal most segment and combine inserting motion of some or all of the segments, as illustrated in frame 5.

There are multiple potential reasons why the user might want to choose some of these options. First, by “borrowing” insert motion from other segments, some of the segments could be constructed with fixed lengths. This reduces the need for segments to telescope inside of each other, and therefore reduces the overall wall thickness. It also reduces the number of insertion degrees-of-freedom needed. Also, by combining the insert motion from several segments, the effective insert range-of-motion for an individual segment can be maximized. In a constrained space such as the vasculature, the operator may likely be interested in “steering” the most distal section while having as much effective insertion range as possible. It would simplify and speed up the workflow to not have to stop and follow with the other segments.

In other embodiments, the “follow” mode may be carried out using a robotic system that includes a flexible elongated member (e.g., a guidewire), a first member (e.g., the catheter 61a) disposed around the flexible elongated member, and a second member (e.g., the sheath 62a) disposed around the first member. The flexible elongated member may have a pre-formed (e.g., pre-bent) configuration. In some embodiments, the flexible elongated member may be positioned inside a body. Such may be accomplished using a drive mechanism that is configured to position (e.g., advance, retract, rotate, etc.) the flexible elongated member. In one example, the positioning of the flexible elongated member comprises advancing the flexible elongated member so that its distal end passes through an opening in the body.

Next, the first member is relaxed so that it has sufficient flexibility that will allow the first member to be guided by the flexible elongated member (that is relatively more rigid than the relaxed first member). In some embodiments, the relaxing of the first member may be accomplished by releasing tension in wires that are inside the first member, wherein the wires are configured to bend the first member or to maintain the first member in a bent configuration. After the first member is relaxed, the first member may then be advanced distally relative to the flexible elongated member. The flexible elongated member, while being flexible, has sufficient rigidity to guide the relaxed first member as the first member is advanced over it. The first member may be advanced until its distal end also passes through the opening in the body.

In some embodiments, the second member may also be relaxed so that it has sufficient flexibility that will allow the second member to be guided by the flexible elongated member (that is relatively more rigid than the relaxed second member), and/or by the first member. In some embodiments, the relaxing of the second member may be accomplished by releasing tension in wires that are inside the second member, wherein the wires are configured to bend the second member or to maintain the second member in a bent configuration. After the second member is relaxed, the second member may then be advanced distally relative to the flexible elongated member. The flexible elongated member, while being flexible, has sufficient rigidity to guide the relaxed second member as the second member is advanced over it. The second member may be advanced until its distal end also passes through the opening in the body. In other embodiments, instead of advancing the second member after the first member, both the first member and the second member may be advanced simultaneously (e.g., using a drive mechanism) so that they move together as a unit. In further embodiments, the acts of advancing the flexible elongated member, the first member, and the second member may be repeated until a distal end of the flexible elongated member, the first member, or the second member has passed through an opening in a body.

In the above embodiments, tension in pull wires in the second elongated member is released to make it more flexible than the first elongated member, and the second elongated member is then advanced over the first elongated member while allowing the first elongated member to guide the second elongated member. In other embodiments, the tension in the pull wires in the first elongated member may be released to make it more flexible than the second elongated member. In such cases, the more flexible first elongated member may then be advanced inside the more rigid second elongated member, thereby allowing the shape of the second elongated member to guide the advancement of the first elongated member. In either case, the more rigid elongated member may be locked into shape by maintaining the tension in the pull wires.

In some of the embodiments described herein, the flexible elongated member may be a guidewire, wherein the guidewire may have a circular cross section, or any of other cross-sectional shapes. Also, in other embodiments, the guidewire may have a tubular configuration. In still other embodiments, instead of a guidewire, the flexible elongated member may be the member 26. In further embodiments, the robotic system may further include a mechanism for controlling and/or maintaining the preformed configuration of the guidewire. In some embodiments, such mechanism may include one or more steering wires coupled to a distal end of the guidewire. In other embodiments, such mechanism may be the catheter 61a, the sheath 62a, or both. In particular, one or both of the catheter 61a and the sheath 62a may be stiffened (e.g., by applying tension to one or more wires inside the catheter 61a and/or the sheath 62a). The stiffened catheter 61a and/or the sheath 62a may then be used to provide support for the guidewire.

Also, in some of the embodiments described herein, any movement of the elongate member 26, the catheter 61a, and/or the sheath 62a may be accomplished robotically using a drive assembly. In some embodiments, the drive assembly is configured to receive a control signal from a processor, and actuate one or more driveable elements to move the elongate member 26, the catheter 61a, and/or the sheath 62a.

It should be noted that the driving modes for the system are not limited to the examples discussed, and that the system may provide other driving modes in other embodiments.

V. Treatment Methods

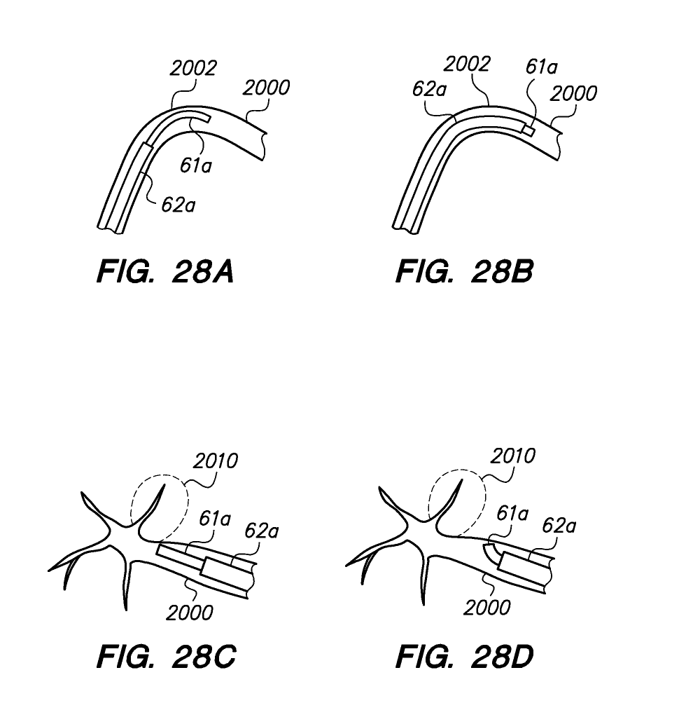

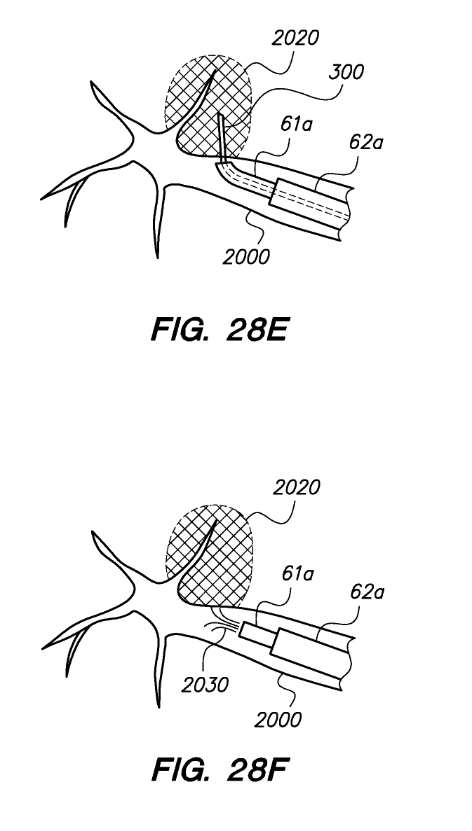

FIGS. 28A-28F illustrate a method of treating tissue at a liver using the robotic system 10 in accordance with some embodiments. First, the robotic system 10 is setup by placing the catheter 61 into the lumen of the sheath 62, and by placing the elongate member 26 into the lumen of the catheter 61. Next, an incision is then made at a patient's skin, and the distal end of the catheter 61 is then inserted into the patient through the incision. In particular, the distal end of the catheter 61 is placed inside a vessel 2000 (e.g., a vein or an artery) of the patient. In some embodiments, the liver may be accessed from the femoral vein or femoral artery from either groin. In other embodiments, the liver may be accessed from the right sub-clavin in vein or the right jugular vein. In some embodiments, the initial insertion of the catheter 61 into the patient may be performed manually. In other embodiments, the initial insertion of the catheter 61 may be performed robotically using the system 10. In such cases, the user may enter a command at the workstation 2, which then generates a user signal in response thereto. The user signal is transmitted to a controller, which then generates a control signal in response to the user signal. The control signal is transmitted to the driver to drive the catheter 61 so that it advances distally into the patient. In some embodiments, while the catheter 61 is being inserted into the patient, the distal end 300 of the elongate member 26 may be housed within the lumen of the catheter 61. In other embodiments, the distal end 300 of the elongate member 26 may extend out of the lumen of the catheter 61 (which the flexible section 320 of the elongate member 26 is housed within the lumen of the catheter 61) as the catheter 61 is being inserted. In such cases, the sharp distal tip of the elongate member 26 may facilitate insertion through the patient's skin. In other embodiments, the tip of the elongate member 26 may not be sharp enough, or the distal section of the elongate member 26 may not be stiff enough, to puncture the patient's skin. In such cases, a separate tool may be used to create an incision at the patient's skin first, as discussed.

In some embodiments, after the catheter 61a is placed inside the patient, the sheath 62a may be advanced distally over the catheter 61a. Alternatively, both the catheter 61a and the sheath 62a may be advanced simultaneously to enter into the patient.

Once the catheter 61a and the sheath 62a are inserted into the patient, they can be driven to advance through the vasculature of the patient. At sections of the vessel 2000 that are relatively straight, both the catheter 61a and the sheath 62a may be driven so that they move as one unit. Occasionally, the catheter 61a and/or the sheath 62a may reach a section of the vessel 2000 that has a bend (e.g., a sharp bend). In such cases, the catheter 61a and the sheath 62a may be driven in a telescopic manner to advance past the bend.

FIGS. 28A-28B illustrate such telescopic technique for advancing the sheath 62a and the catheter 61a over a bend 2002 along a length of the vessel 2000. In this technique, the catheter 61a is positioned with its distal articulation section traversing the bend 2002 and it is locked in this position (FIG. 28A). Next, the sheath 62a is advanced over the catheter 61a (FIG. 28B), and the catheter 61a acts as a rail held in a fixed shape for the sheath 62a to glide over. As the sheath 62a is advanced further, sections with higher bending stiffness on the sheath 62a will pass over the articulated section of the catheter 61a, putting an increase load on the catheter 61a. The increase in load on the catheter 61a may tend to straighten the catheter 61a. In some embodiments, the drive assembly of the robotic system 10 maintains the bent shape of the catheter 61a by tightening the control wire(s), which has the effect of stiffening the catheter 61a. In some embodiments, the robotic system 10 is configured to detect the increased load on the control wires (due to the placement of the sheath 62a over the catheter 61a) to be detected. The operator, or the robotic system 10, can then apply an equal counteracting load on all the control wires of the catheter 61a to ensure that its bent shape is maintained while the sheath 62a is advanced over the bend. In other embodiments, the sheath 62a may be extremely flexible so that it does not put any significant load on the catheter 61a as the sheath 62a is advanced over the catheter 61a, and/or distort the anatomy.

Once the distal end of the catheter 61a reaches the target location (FIG. 28C), the distal end of the catheter 61a may be steered to create a bend so that the distal opening at the catheter 61a faces towards a tissue 2010 that is desired to be treated (FIG. 28D). The steering of the distal end of the catheter 61a may be accomplished by receiving a user input at the workstation 2, which generates a user signal in response to the user input. The user signal is transmitted to the controller, which then generates a control signal in response to the user signal. The control signal causes the drive assembly to apply tension to one or more wires inside the catheter 61a to thereby bend the distal end of the catheter 61a at the desired direction.

Next, the distal end 300 of the elongate member 26 is deployed out of the lumen of the catheter 61a by advancing the elongate member 26 distally (FIG. 28E). This may be accomplished robotically using the manipulator 24, and/or manually. The sharp distal tip of the elongate member 26 allows the distal end 300 to penetrate into the target tissue 2010. Also, the flexible section 320 of the elongate member 26 allows the elongate member 26 to follow the curvature of the catheter 61a as the elongate member 26 is advanced out of the lumen of the catheter 61a. In some embodiments, the distal advancement of the elongate member 26 may be accomplished by receiving a user input at the workstation 2, which generates a user signal in response to the user input. The user signal is transmitted to the controller, which then generates a control signal in response to the user signal. The control signal causes the elongate member manipulator 24 to turn its roller(s) to thereby advance the elongate member 26 distally.

After the distal end 300 of the elongate member 26 is desirably positioned, the RF generator 350 is then activated to cause the distal end 300 to deliver RF ablation energy to treat the target tissue 2010. In some embodiments, if the system 10 includes the return electrode 352 that is placed on the patient's skin, the system 10 then delivers the energy in a monopolar configuration. In other embodiments, if the elongate member 26 includes the two electrodes 370a, 370b, the system 10 may then deliver the energy in a bipolar configuration. The energy is delivered to the target tissue 2010 for a certain duration until a lesion 2020 is created at the target site (FIG. 28E).

In some embodiments, while energy is being delivered by the elongate member 26, cooling fluid may be delivered to the target site through the lumen in the elongate member 26, and out of the distal port 310 and/or side port(s) 312 at the elongate member 26. The cooling fluid allows energy to be delivered to the target tissue in a desired manner so that a lesion 3020 of certain desired size may be created. In other embodiments, the delivery of cooling fluid is optional, and the method does not include the act of delivering cooling fluid.

After the lesion 3020 has been created, the elongate member 26 may be removed from the catheter 61a, and a substance 2030 may then be delivered to the target site through the lumen of the catheter 61a (FIG. 28F). In some embodiments, the removal of the elongate member 26 from the catheter 61a may be accomplished by receiving a user input at the workstation 2, which generates a user signal in response to the user input. The user signal is transmitted to the controller, which then generates a control signal in response to the user signal. The control signal causes the elongate member manipulator 24 to turn its roller(s) to thereby retract the elongate member 26 proximally until the entire elongate member 26 is out of the lumen of the catheter 61a.

In some embodiments, the substance 2030 may be an embolic material for blocking supply of blood to the target site. In other embodiments, the substance 2030 may be a drug, such as a chemotherapy drug, for further treating tissue at the target site. In further embodiments, the substance 2030 may be one or more radioactive seeds for further treating tissue at the target site through radiation emitted from the radioactive seed(s). In other embodiments, the delivery of the substance 2030 may be optional, and the method may not include the act of delivering the substance 2030.

In some embodiments, if there is another target tissue (e.g., tumor) that needs to be treated, any or all of the above actions may be repeated. For example, in some embodiments, after the first tumor has been ablated, the distal end of the catheter 61a may be steered to point to another direction, and the elongate member 26 may be deployed out of the catheter 61a again to ablate the second tumor. Also, in other embodiments, the catheter 61a may be moved distally or retracted proximally along the length of the vessel 2000 to reach different target sites.

In other embodiments, instead of the telescopic configuration, the robotic system 10 may be configured to drive the catheter 61a and the sheath 62a in other configurations. For example, in some embodiments, the sheath 62a may be bent and acts as guide for directing the catheter 61a to move in a certain direction. In such cases, the robotic system 10 may be configured to relax the wires in the catheter 61a so that the catheter 61a is flexible as it is advanced distally inside the lumen of the sheath 62a. Also, in other embodiments, the sheath 62a may not be involved in the method. In such cases, the robotic system 10 may be configured to drive the catheter 61a without the sheath 62a to advance the catheter 61a through the vasculature of the patient.

Also, in other embodiments, a guidewire may be used in combination with the catheter 61a and/or the sheath 62a for advancement of the catheter 61a and/or the sheath 62a inside the vessel of the patient. In such cases, the elongate member 26 is not inserted into the catheter 61a. Instead, the guidewire is coupled to the elongate member manipulator 24, and the guidewire is placed inside the lumen of the catheter 61a. The manipulator 24 may then be used to drive the guidewire to advance and/or retract the guidewire. In some cases, the robotic system 10 may advance the guidewire, the catheter 61a, and the sheath 62a in a telescopic configuration, as similarly discussed.

If a guidewire is initially used to access the interior of the patient, the guidewire may be later exchanged for the elongate member 26. For example, in some embodiments, the guidewire may be exchanged for the elongate member 26 after initial access of the main hepatic artery (or vein). After the distal end of the catheter 61a reaches the target site, the guidewire may then be removed from the lumen of the catheter 61a, and decoupled from the elongate member manipulator 24. The proximal end of the elongate member 26 is coupled to the elongate member manipulator 24, and the elongate member 26 is then inserted into the lumen of the catheter 61a. The elongate member manipulator 24 is then used to drive the elongate member 26 distally until the distal end 300 of the elongate member 26 exits out of the distal end of the catheter 61a, as similarly discussed.

In further embodiments, the elongate member 26 may not be needed to treat tissue. For example, in other embodiments, after the distal end of the catheter 61a is desirably placed at a target site, the catheter 61a may then be used to deliver a substance (e.g., an agent, a drug, radioactive seed(s), embolic material, etc.) to treat tissue at the target site without ablating the tissue. In some embodiments, the catheter 61a itself may be directly used to deliver the substance. In other embodiments, another delivery device (e.g., a tube) may be placed inside the lumen of the catheter 61a, and the delivery device is then used to deliver the substance. In such cases, the catheter 61a is used indirectly for the delivery of the substance.

In some embodiments, during the treatment method, a localization technique may be employed to determine a location of the instrument inside the patient's body. The term “localization” is used in the art in reference to systems for determining and/or monitoring the position of objects, such as medical instruments, in a reference coordinate system. In one embodiment, the instrument localization software is a proprietary module packaged with an off-the-shelf or custom instrument position tracking system, which may be capable of providing not only real-time or near real-time positional information, such as X-Y-Z coordinates in a Cartesian coordinate system, but also orientation information relative to a given coordinate axis or system. For example, such systems can employ an electromagnetic based system (e.g., using electromagnetic coils inside a device or catheter body). Other systems utilize potential difference or voltage, as measured between a conductive sensor located on the pertinent instrument and conductive portions of sets of patches placed against the skin, to determine position and/or orientation. In another similar embodiment, one or more conductive rings may be electronically connected to a potential-difference-based localization/orientation system, along with multiple sets, preferably three sets, of conductive skin patches, to provide localization and/or orientation data. Additionally, “Fiberoptic Bragg grating” (“FBG”) sensors may be used to not only determine position and orientation data but also shape data along the entire length of a catheter or shapeable instrument. In other embodiments, imaging techniques may be employed to determine a location of the instrument inside the patient's body. For examples, x-ray, ultrasound, computed tomography, MRI, etc., may be used in some embodiments.

In other embodiments not comprising a localization system to determine the position of various components, kinematic and/or geometric relationships between various components of the system may be utilized to predict the position of one component relative to the position of another. Some embodiments may utilize both localization data and kinematic and/or geometric relationships to determine the positions of various components. The use of localization and shape technology is disclosed in detail in U.S. patent application Ser. Nos. 11/690,116, 11/176,598, 12/012,795, 12/106,254, 12/507,727, 12/822,876, 12/823,012, and 12/823,032, the entirety of all of which is incorporated by reference herein for all purposes.

Also, in one or more embodiments described herein, the system may further include a sterile barrier positioned between the drive assembly and the elongate member holder, wherein the drive assembly is configured to transfer rotational motion, rotational motion, or both, across the sterile barrier to the rotary members to generate the corresponding linear motion of the elongate member along the longitudinal axis of the elongate member, rotational motion of the elongate member about the longitudinal axis, or both linear motion and rotational motion.

As illustrated in the above embodiments, the robotic technique and system 10 for treating liver tissue is advantagoues because it allows the ablation device to reach certain part(s) of the liver through the vessel that may otherwise not be possible to reach using conventional rigid ablation probe. For example, in some embodiments, using the robotic system 10 and the above technique may allow the distal end of the elongate member 26 to reach the lobus quatratus or the lobus spigelii of the liver, which may not be possible to reach by conventional ablation probe. Also, using the elongate member manipulator 24 to position the elongate member 26 is advantageous because it allows accurate positioning of the distal end 300 of the elongate member 26.

VI. Other Clinical Applications

The different driving modes and/or different combinations of driving modes are advantageous because they allow an elongate instrument (catheter 61a, sheath 61b, elongate member 26, or any combination thereof) to access any part of the vasculature. Thus, embodiments of the system described herein may have a wide variety of applications. In some embodiments, embodiments of the system described herein may be used to treat thoracic aneurysm, thoracoabdominal aortic aneurysm, abdominal aortic aneurysm, isolated common iliac aneurysm, visceral arteries aneurysm, or other types of aneurysms. In other embodiments, embodiments of the system described herein may be used to get across any occlusion inside a patient's body. In other embodiments, embodiments of the system described herein may be used to perform contralateral gait cannulation, fenestrated endograft cannulation (e.g., cannulation of an aortic branch), cannulation of internal iliac arteries, cannulation of superior mesenteric artery (SMA), cannulation of celiac, and cannulation of any vessel (artery or vein). In further embodiments, embodiments of the system described herein may be used to perform carotid artery stenting, wherein the tubular member may be controlled to navigate the aortic arch, which may involve complex arch anatomy. In still further embodiments, embodiments of the system described herein may be used to navigate complex iliac bifurcations.

In addition, in some embodiments, embodiments of the system described herein may be used to deliver a wide variety of devices within a patient's body, including but not limited to: stent (e.g., placing a stent in any part of a vasculature, such as the renal artery), balloon, vaso-occlusive coils, any device that may be delivered over a wire, an ultrasound device (e.g., for imaging and/or treatment), a laser, any energy delivery devices (e.g., RF electrode(s)), etc. In other embodiments, embodiments of the system described herein may be used to deliver any substance into a patient's body, including but not limited to contrast (e.g., for viewing under fluoroscope), drug, medication, blood, etc. In one implementation, after the catheter 61a (leader) is placed at a desired position inside the patient, the catheter 61a and the elongate member 26 may be removed, leaving the sheath 61b to provide a conduit for delivery of any device or substance. In another implementation, the elongate member 26 may be removed, leaving the catheter 61a to provide a conduit for delivery of any device or substance. In further embodiments, the elongate member 26 itself may be used to deliver any device or substance.

In further embodiments, embodiments of the system described herein may be used to access renal artery for treating hypertension, to treat uterine artery fibroids, atherosclerosis, and any peripheral artery disease. Also, in other embodiments, embodiments of the system described herein may be used to access the heart. In some embodiments, embodiments of the system may also be used to deliver drug or gene therapy.

In still further embodiments, embodiments of the system described herein may be used to access any internal region of a patient that is not considered a part of the vasculature. For example, in some cases, embodiments of the system described herein may be used to access any part of a digestive system, including but not limited to the esophagus, liver, stomach, colon, urinary tract, etc. In other embodiments, embodiments of the system described herein may be used to access any part of a respiratory system, including but not limited to the bronchus, the lung, etc.

In some embodiments, embodiments of the system described herein may be used to treat a leg that is not getting enough blood. In such cases, the tubular member may access the femoral artery percutaneously, and is steered to the aorta iliac bifurcation, and to the left iliac. Alternatively, the tubular member may be used to access the right iliac. In one implementation, to access the right iliac, the drive assembly may be mounted to the opposite side of the bed (i.e., opposite from the side where the drive assembly is mounted in FIG. 1). In other embodiments, instead of accessing the inside of the patient through the leg, the system may access the inside of the patient through the arm (e.g., for accessing the heart).

In any of the clinical applications mentioned herein, the telescopic configuration of the catheter 61a and the sheath 61b (and optionally the elongate member 26) may be used to get past any curved passage way in the body. For example, in any of the clinical applications mentioned above, a guidewire placed inside the catheter 61a may be advanced first, and then followed by the catheter 61a, and then the sheath 61b, in order to advance the catheter 61a and the sheath 61b distally past a curved (e.g., a tight curved) passage way. Once a target location is reached, the guidewire may be removed from the catheter 61a, and the elongate member 26 may optionally be inserted into the lumen of the catheter 61a. The elongate member 26 is then advanced distally until its distal exits from the distal opening at the catheter 61a. In other embodiments, the catheter 61a may be advanced first, and then followed by the sheath 61b, in order to advance the catheter 61a and the sheath 61b distally past a curved (e.g., a tight curved) passage way. In still further embodiments, the guidewire may be advanced first, and then followed by the catheter 61a the sheath 61b (i.e., simultaneously), in order to advance the catheter 61a and the sheath 61b distally past a curved (e.g., a tight curved) passage way.

Each of the individual variations described and illustrated herein has discrete components and features which may be readily separated from or combined with the features of any of the other variations. Modifications may be made to adapt a particular situation, material, composition of matter, process, process act(s) or step(s) to the objective(s), spirit or scope of the present application. Also, any of the features described herein with reference to a robotic system is not limited to being implemented in a robotic system, and may be implemented in any non-robotic system, such as a device operated manually.

Methods recited herein may be carried out in any order of the recited events which is logically possible, as well as the recited order of events. Furthermore, where a range of values is provided, every intervening value between the upper and lower limit of that range and any other stated or intervening value in that stated range is encompassed. Also, any optional feature described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that described herein (in which case what is present herein shall prevail). The referenced items are provided solely for their disclosure prior to the filing date of the present application. Nothing herein is to be construed as an admission that any claimed invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art in the field of this application.

Although particular embodiments have been shown and described, it will be understood that they are not intended to limit the claimed inventions, and it will be obvious to those skilled in the art having the benefit of this disclosure that various changes and modifications may be made. The specification and drawings are, accordingly, to be regarded in an illustrative rather than restrictive sense. The claimed inventions are intended to cover alternatives, modifications, and equivalents.

1. A robotic system, comprising:
an elongate member comprising a flexible proximal portion, a distal rigid needle attached to the proximal portion, and an operative element for treating tissue, the needle having a distal port; and
an elongate member holder having first and second rotary members configured to hold and manipulate the proximal portion of the elongate member, wherein the first rotary member defines a first rotational axis, and the second rotary member defines a second rotational axis,
wherein the first and second rotary members are moveable relative to each other in opposite rotational directions about their respective axes to generate a corresponding linear motion of the elongate member along a longitudinal axis of the elongate member when the elongate member is held by the rotary members; and
wherein at least one of the first and second rotary members is moveable in a linear direction along its rotational axis to generate a corresponding rotational motion of the elongate member about the longitudinal axis of the elongate member when the elongate member is held by the rotary members.

2. The robotic system of claim 1, wherein operative element comprises a portion of the needle.

3. The robotic system of claim 1, wherein the needle further comprises a plurality of side ports disposed along a length of the needle.

4. The robotic system of claim 1, wherein the flexible proximal portion of the elongate member comprises a tubular member having a solid wall, wherein a portion of the wall is cutout.

5. The robotic system of claim 4, wherein the cutout has a spiral configuration.

6. The robotic system of claim 1, further comprising a drive assembly operatively coupled to the first and second rotary members for actuation of the first and second rotary members, wherein the elongate member holder is releasably coupled to the drive assembly.

7. The robotic system of claim 6, further comprising a sterile barrier positioned between the drive assembly and the elongate member holder, wherein the drive assembly is configured to transfer a respective rotational motion across the sterile barrier to at least one of the rotary members.

8. The robotic system of claim 6, further comprising a sterile barrier positioned between the drive assembly and the elongate member holder, wherein the drive assembly is configured to transfer a respective linear motion across the sterile barrier to at least one of the rotary members.

9. The robotic system of claim 6, wherein the drive assembly is configured to simultaneously actuate one or both of the rotary members in respective rotational and linear motions.

10. The robotic system of claim 6, wherein the drive assembly is configured to simultaneously actuate one or both of the rotary members in respective rotational and linear motions at different respective rates.

11. The robotic system of claim 6, wherein the drive assembly is configured to provide rotational actuation of the rotary members for translating the elongate member, and linear actuation of the rotary members for rotating the elongate member, respectively, when the elongate member is held by the rotary members; and
wherein the rotary members are configured to maintain engagement with the elongate member between a transition from translating the elongate member to rotating the elongate member.

12. The robotic system of claim 1, wherein the first and second rotary members comprise first and second feed rollers.

13. The robotic system of claim 12, wherein the first feed roller is motor driven and the second feed roller is passive.

14. The robotic system of claim 1, wherein the first and second rotary members comprise respective flexible members with respective engagement surfaces.

15. The robotic system of claim 14, wherein the first rotary member is motor driven and the second rotary member is passive.

16. The robotic system of claim 14, wherein the flexible members comprise respective feed belts.

17. The robotic system of claim 1, wherein the first and second rotary members are each moveable relative to each other in a linear direction along their respective rotational axes to generate the corresponding rotational motion of the elongate member.

18. The robotic system of claim 1, further comprising:
a second elongate member circumferentially disposed around at least a portion of the first elongate member; and
a drive assembly operatively coupled to the second elongate member for moving the second elongate member.

19. The robotic system of claim 18, further comprising:
a third elongate member circumferentially disposed around at least a portion of the second elongate member;
wherein the drive assembly is also operatively coupled to the third elongate member for moving the third elongate member.

20. The robotic system of claim 1, wherein one of the first and second rotary members is a passive rotary member, and the robotic system further comprises a slip-sensor coupled to the passive rotary member.

21. A method of manipulating an elongate member in at least two degrees of freedom, comprising:
holding an elongate member between two rotary members that define respective rotational axes, the elongate member having a flexible proximal portion, a distal rigid needle attached to the proximal portion, and an operative element for delivering energy, the needle having a distal port;
actuating at least one of the rotary members in a rotational direction about its rotational axis to generate a corresponding linear motion of the elongate member along a longitudinal axis of the elongate member; and
actuating at least one of the rotary members in a linear direction along its rotational axis to generate a corresponding rotational motion of the elongate member about the longitudinal axis of the elongate member.

22. The method of claim 21, wherein the rotary members comprise feed belts.

23. The method of claim 21, wherein the acts of actuating are performed simultaneously.

24. The method of claim 21, wherein the acts of actuating are performed at different respective rates.

25. The method of claim 21, wherein the acts of actuating are performed separately, and wherein between the acts or actuating, the rotary members maintain engagement with the elongate member.

26. The method of claim 21, further comprising loading the elongate member by separating the two rotary members, and placing the elongate member on a surface of one of the two rotary members.

27. The method of claim 21, wherein the rotary members comprises a first flexible member with a first engagement surface for direct engagement with the elongate member, and the second rotary member comprises a second flexible member with a second engagement surface for direct engagement with the elongate member.

28. The method of claim 21, wherein the acts of actuating are performed to position the elongate member at a liver.

29. The method of claim 21, wherein the energy comprises RF energy, and the method further comprises:
delivering the RF energy to tissue at the liver using the operative element; and
using the distal port of the needle to deliver fluid to control the delivering of the RF energy.

30. The method of claim 21, further comprising delivering a substance at the liver using the needle.

31. The method of claim 30, wherein the substance comprises a drug, an agent, an embolic, or a radioactive seed.

32. The method of claim 21, further comprising using the needle to treat tissue at a lobus quatratus of the liver.

33. The method of claim 21, further comprising using the needle to treat tissue at a lobus spigelii of the liver.
